(19) United States
(12) Patent Application Publication
(10) Pub. No.: US 2025/0052763 A1
(43) Pub. Date: Feb. 13, 2025

(54) COMPOSITIONS AND METHODS FOR IDENTIFYING LIGANDS OF ODORANT RECEPTORS

(71) Applicant: Duke University, Durham, NC (US)

(72) Inventors: Hiroaki Matsunami, Durham, NC (US); Aashutosh Vihani, Durham, NC (US)

(21) Appl. No.: 18/718,238

(22) PCT Filed: Dec. 14, 2022
(86) PCT No.: PCT/US22/52771

§ 371 Date: Jun. 10, 2024

Related U.S. Application Data
(60) Provisional application No. 63/289,337, filed on Dec. 14, 2021.

(51) Int. Cl. G01N 33/68; G16B 15/30; G16B 35/00

(52) U.S. Cl. CPC G01N 33/6845; G16B 15/30; G16B 35/00

(57) ABSTRACT

The present invention relates to compositions and methods for identifying odorant-odorant receptor interactions. In particular, the present invention relates to methods for identifying odorant receptor-odorant interactions based on odorant receptor amino acid sequence and other properties of odorant receptors and odorants. The methods provide, in part, for the broad surveying of OR responses, using an in vivo strategy, against a diverse panel of 10 odorants, followed by using the resulting interaction profiles to uncover relationships between OR responses, odorants, odor molecular properties, and OR sequences.

### CROSS REFERENCE TO RELATED APPLICATIONS

This application claims priority to provisional application 63/289,337, filed Dec. 14, 2021, which is herein incorporated by reference in its entirety.

### STATEMENT REGARDING FEDERALLY SPONSORED RESEARCH OR DEVELOPMENT

This invention was made with Government support under Federal Grant nos. DC014423 and DC16224 awarded by the National Institute on Deafness & Other Communication Disorders (NIH/NIDCD) and Federal Grant nos. 1556207 and 1555919 awarded by the National Science Foundation. The Federal Government has certain rights to this invention.

### FIELD OF THE INVENTION

The present invention relates to compositions and methods for identifying odorant-odorant receptor interactions. In particular, the present invention relates to methods for identifying odorant receptor-odorant interactions based on odorant receptor amino acid sequence and other properties of odorant receptors and odorants.

### BACKGROUND OF THE INVENTION

Olfactory dysfunction arises from a variety of causes and profoundly influences a patient's quality of life. Approximately 2 million Americans experience some type of olfactory dysfunction. Studies show that olfactory dysfunction affects at least 1% of the population under the age of 65 years, and well over 50% of the population older than 65 years. The sense of smell determines the flavor of foods and beverages and serves as an early warning system for the detection of environmental hazards, such as spoiled food, leaking natural gas, smoke, or airborne pollutants. The losses or distortions of smell sensation can adversely influence food preference, food intake and appetite.

Olfactory disorders are classified as follows: 1) anosmia: inability to detect qualitative olfactory sensations (e.g., absence of smell function), 2) partial anosmia: ability to perceive some, but not all, odorants, 3) hyposmia or microsmia: decreased sensitivity to odorants, 4) hyperosmia: abnormally acute smell function, 5) dysosmia (cacosmia or parosmia): distorted or perverted smell perception or odorant stimulation, 6) phantosmia: dysosmic sensation perceived in the absence of an odor stimulus (a.k.a. olfactory hallucination), and 7) olfactory agnosia: inability to recognize an odor sensation.

Olfactory dysfunction is further classified as 1) conductive or transport impairments from obstruction of nasal passages (e.g., chronic nasal inflammation, polyposis, etc.), 2) sensorineural impairments from damage to neuroepithelium (e.g., viral infection, airborne toxins, etc.), 3) central olfactory neural impairment from central nervous system damage (e.g., tumors, masses impacting on olfactory tract, neurodegenerative disorders, etc.). These categories are not mutually exclusive. For example, viruses can cause damage to the olfactory neuroepithelium and they may also be transported into the central nervous system via the olfactory nerve causing damage to the central elements of the olfactory system.

Smelling abilities are initially determined by neurons in the olfactory epithelium, the olfactory sensory neurons (hereinafter “olfactory neurons). In olfactory neurons, odorant receptor (hereinafter “OR”) proteins, members of the G-protein coupled receptor (hereinafter “GPCR”) superfamily, are synthesized in the endoplasmic reticulum, transported, and eventually concentrated at the cell surface membrane of the cilia at the tip of the dendrite. Considering that ORs have roles in target recognition of developing olfactory axons, OR proteins are also present at axon terminals (see, e.g., Mombaerts, P., (1996) Cell 87, 675-686; Wang, F., et al. (1998) Cell 93, 47-60; each herein incorporated by reference in their entireties). In rodents, odorants are transduced by as many as 1000 different ORs encoded by a multigene family (see, e.g., Axel, R. (1995) Sci Am 1273, 154-159; Buck, L., and Axel, R. (1991) Cell 65, 175-187; Firestein, S. (2001) Nature 413, 211-218; Mombaerts, P. (1999) Annu Rev Neurosci 22, 487-509; Young, J. M., et al., (2002) Hum Mol Genet 11, 535-546; Zhang, X., and Firestein, S. (2002) Nat Neurosci 5, 124-133; each herein incorporated by reference in their entirety). Each olfactory neuron expresses only one type of the OR, forming the cellular basis of odorant discrimination by olfactory neurons (see, e.g., Lewcock, J. W., and Reed, R. R. (2004) Proc Natl Acad Sci USA; Malnic, B., et al., (1999) Cell 96, 713-723; Serizawa, S., et al., (2003) Science 302, 2088-2094; each herein incorporated by reference in their entirety).

What is needed is a better understanding of olfactory sensation. What is further needed is a better understanding of odorant receptor function and odorant receptor-odorant interactions.

### SUMMARY OF THE INVENTION

The present disclosure is based, in part, on the discovery by the inventors of a novel method for identifying novel ligands that bind odorant receptors. The methods provide, in part, for the broad surveying of OR responses, using an in vivo strategy, against a diverse panel of odorants, followed by using the resulting interaction profiles to uncover relationships between OR responses, odorants, odor molecular properties, and OR sequences.

Accordingly, in some embodiments, provided herein is a method of identifying a ligand for an odorant receptor, comprising: a) determining the sequence of transmembrane domains 4 and 5 of the receptor; and b) identifying the receptor as likely to bind to a sulfurous odorant when one or more of a E or R amino acid at position 137, a P amino acid at position 138, A N amino acid at position 141, a C amino acid at position 148, a T amino acid at position 149, a G amino acid at position 151, a G amino acid at position 152, a A amino acid at position 153, a D amino acid at position 155, a G amino acid at position 156, a F amino acid at position 157, a M amino acid at position 158, a Y amino acid at position 195, a N amino acid at position 196, a T amino acid at position 197, a F amino acid at position 198, an M amino acid at position 199, a T, V or N amino acid at position 200, a A amino acid at position 201, a C or D amino acid at position 202, a C amino acid at position 203, a A or Y amino acid at position 205, an M amino acid at position 206, a N amino acid at position 210, a V amino acid at position 213, a L amino acid at position 214, a S amino acid at position 215, a S amino acid at position 219, a S amino acid at position 220, and an L amino acid at position 223 is present in the receptor.

Further embodiments provide a method of identifying a ligand for an odorant receptor, comprising: a) determining the sequence of transmembrane domains 4 and 5 of the receptor; and b) identifying the receptor as likely to bind to a carboxylic acid odorant when one or more of an R amino acid at position 150, a I amino acid at position 155, and a D amino acid at position 210 is present in the receptor.

Also provided is a method of identifying a ligand for an odorant receptor, comprising: a) determining the sequence of transmembrane domains 4 and 5 of the receptor; and b) identifying the receptor as likely to bind to an aldehyde acid odorant when one or more of a I or V amino acid at position 138, an R amino acid at position 139, a Y amino acid at position 141, a V amino acid at position 148, a C amino acid at position 149, a S amino acid at position 150, a T amino acid at position 151, an N amino acid at position 155, a W amino acid at position 156, a K or A amino acid at position 158, an A or P amino acid at position 195, a A amino acid at position 196, a E amino acid at position 197, an E or N amino acid at position 198, a P amino acid at position 199, a S amino acid at position 202, a V or R amino acid at position 203, a S amino acid at position 205, a C, N or V amino acid at position 209, a F amino acid at position 212, a A or G amino acid at position 215, a Y amino acid at position 220, and a H amino acid at position 224 is present in the receptor.

Additional embodiments provide a method of identifying a ligand for an odorant receptor, comprising: a) determining the sequence of transmembrane domains 4 and 5 of the receptor; and b) identifying the receptor as likely to bind to an alcohol odorant when one or more of a F, G or E amino acid at position 137, a L, P or H amino acid at position 138, a T, A or K amino acid at position 139, a R, Q or S amino acid at position 142, a L amino acid at position 143, a T or P amino acid at position 147, a S amino acid at position 148, a M or G amino acid at position 149, a A or S amino acid at position 150, a A or S amino acid at position 151, a C amino acid at position 152, a E or C amino acid at position 154, a E amino acid at position 155, a G amino acid at position 157, a H, Q, E, C, or F amino acid at position 158, a E or V amino acid at position 155, a P, K or Q amino acid at position 195, a W or M amino acid at position 197, a S or Q amino acid at position 198, a P or N amino acid at position 199, a Y amino acid at position 200, a C or Y amino acid at position 201, a G, S or F amino acid at position 202, an G, H, A or I amino acid at position 203, a V or N amino acid at position 204, a N amino acid at position 206, an A, I or F amino acid at position 207, a P amino acid at position 208, an A amino acid at position 209, a A or V amino acid at position 210, a M amino acid at position 211, an T, V, S or I amino acid at position 212, a G amino acid at position 215, a V or N amino acid at position 216, a W amino acid at position 219, a R amino acid at position 220, a F amino acid at position 222, a V amino acid at position 223, and a C amino acid at position 210 is present in the receptor.

Further provided is a method of identifying a ligand for an odorant receptor, comprising: a) determining the sequence of transmembrane domains 4 and 5 of the receptor; and b) identifying the receptor as likely to bind to a ketone odorant when one or more of a L or H amino acid at position 138, a H or R amino acid at position 141, a S or N amino acid at position 142, a M or G amino acid at position 146, a V amino acid at position 149, a M or Y amino acid at position 150, an E, W or G amino acid at position 154, a A or P amino acid at position 155, a P or W amino acid at position 156, a A amino acid at position 158, an M or P amino acid at position 195, a A amino acid at position 196, a G amino acid at position 198, a P or C amino acid at position 199, a F amino acid at position 202, an M or V amino acid at position 203, a P, M or G amino acid at position 205, a H or P amino acid at position 206, a I amino acid at position 207, a V amino acid at position 209, a T amino acid at position 211, a T amino acid at position 213, a F amino acid at position 215, a T amino acid at position 217, a Q amino acid at position 220, a M amino acid at position 223, and a N amino acid at position 224 is present in the receptor.

Additionally provided is a method of identifying a ligand for an odorant receptor, comprising: a) determining the sequence of transmembrane domains 4 and 5 of the receptor; and b) identifying the receptor as likely to bind to a pyridine or pyrazine odorant when one or more of a M amino acid at position 137, an C or A amino acid at position 138, a R amino acid at position 140, a V amino acid at position 143, a V amino acid at position 147, a G amino acid at position 148, a N amino acid at position 152, a T amino acid at position 153, a D amino acid at position 154, a Y or S amino acid at position 155, a G amino acid at position 158, an P, H or Y amino acid at position 195, an F amino acid at position 196, a S amino acid at position 197, a P amino acid at position 209, a T amino acid at position 201, a M amino acid at position 202, a F or L amino acid at position 203, a F amino acid at position 204, a Y or G amino acid at position 205 is present in said receptor, a P amino acid at position 206, an E amino acid at position 207, a W amino acid at position 208, a C amino acid at position 213, a A amino acid at position 216, and a C or L amino acid at position 219 is present in the receptor.

Certain aspects provide a method of identifying a ligand for an odorant receptor, comprising: a) determining the sequence of transmembrane domains 4 and 5 of the receptor; and b) identifying the receptor as likely to bind to ester odorant when one or more of a K amino acid at position 137, a I amino acid at position 138, a S amino acid at position 140, a G amino acid at position 148, a N amino acid at position 152, a T amino acid at position 154, a N or P amino acid at position 155, a Y amino acid at position 195, a Y amino acid at position 197, a T amino acid at position 198, a G amino acid at position 201, a S amino acid at position 202, a V amino acid at position 210, a M amino acid at position 213, a T amino acid at position 215, a G amino acid at position 219, a I amino acid at position 220, and a N amino acid at position 223 is present in the receptor.

Other embodiments provide a method of identifying a ligand for an odorant receptor, comprising: a) determining the sequence of transmembrane domains 4 and 5 of the receptor; and b) identifying the receptor as likely to bind to a thiazole or thiazoline odorant when one or more of a S amino acid at position 141, a F amino acid at position 142, a M amino acid at position 144, a C amino acid at position 151, an T amino acid at position 153, a H or D amino acid at position 154, a E amino acid at position 198, a S amino acid at position 199, a L amino acid at position 203, a K amino acid at position 205, a D or P amino acid at position 206, a I amino acid at position 211, a V amino acid at position 214, a Y amino acid at position 216, a M amino acid at position 220, a S amino acid at position 223, and a D amino acid at position 210 is present in the receptor.

In some embodiments, the one or more amino acids is two or more (e.g., 2, 3, 4, 5, 6, 7, 8, 9, 10, or all) of the amino acids. Certain aspects of the methods are performed in silico and, e.g., displayed on a computer screen.

In exemplary embodiments, the method further comprises the step of screening odorants and odorant receptor pairs identified in said method using an vitro assay.

Additional embodiments are described herein.

### DESCRIPTION OF THE FIGURES

FIG. 1 shows data bias and pS6-IP-Seq validation. A, A total of 1811 molecular descriptors were calculated for a total of 4732 small molecules. B, Left, Grantham distances were used to calculate a distance matrix for intact ORs. C, ORs responsive to tested enantiomers were evaluated by heterologous expression. ORs enriched by pS6-TP-Seq against (+)-odorant are colored red while ORs enriched by (−)-odorant are colored blue. ORs enriched by both enantiomers are colored purple.

FIG. 2 shows combinatorial coding and OR tuning. A, Odor responses across the 52 unique odorants, tested at low concentrations, visualized by heatmap. Responses are color coded from negative, to zero, to positive responses in light blue to white to red. The following odorants are abbreviated: 2-methyl-2-thaizoline (2M2T), 2,4,5-trimethyl-4,5-dihydrothiazole (TMT), 2-sec-butyl-4,5-dihydrothiazole (SBT), 2,4,5-trimethylthiazole (nTMT), and 3,4-dehydro-exo-brevicomin (DHB). B, Odor responses across the same 52 odorants visualized by chord plot. Association band thickness corresponds to the number of ORs shared, while color corresponds to overall response similarity across the 375 deorphanized ORs using correlation distance (1-r). C, Z-scored odor response spectra of five example ORs across the 52 tested odorants. Responses were normalized such that each OR is z-scored. D, Histogram of the number of significant odorants per receptor. E, Z-scored PSVs of five example molecular properties across the same set of odorants as B. F, Property responses given by the Pearson's correlation coefficients between the odor responses (B) and PSVs (D) calculated over the 52 odorants in the panel. G, Property response spectra characterized by Pearson's correlation coefficients between the five example ORs and 1811 molecular properties. H, Histogram of the number of significant molecular properties per receptor (n=2967).

FIG. 3 shows pairwise odor similarity comparisons in response and property spaces. A, Pairwise correlation distance measurements between odorants in response space regressed against molecular property space (r=0.29, p=3.4E-27, n=1326). B, LASSO regression as a function of varying the loss function (λ). Large λ values (leftmost edge) generally correspond to the selection of a few weighted molecular properties. C, Pairwise correlation distance measurements between odorants in response space against optimized molecular property space (r=0.34, p=3.2E-37, n=1326). D, Ridge regression results as a function of varying the λ loss function using the optimized set of molecular properties. F, Results of odor pair cross-validation using the XGBoost model framework with default hyperparameters with the optimized set of molecular properties (r=0.31, n=1326).

FIG. 4 shows sequence-function relationships of ORs. A, ORs were split into 90% training and 10% testing sets for 100 repetitions of regularized logistic regression using aligned sequences as predictors. B, Left, the most commonly occurring amino acid was quantified by its percent conservation across ORs and visualized by snake plot. Right, percent conservation distributions were visualized across receptor domains by box-and-whisker plot. C, Regressing the average weight assigned to a position, by regularized logistic regression, by its percent conservation reveals an anti-correlation (r=−0.38, p=6.1E-12). D, Using a SVM classifier with a linear kernel to predict response likelihoods of held-out ORs leads to similar results as logistic regression (logistic regression AUROC=0.70, linear SVM AUROC=0.78). E, ORs sharing response to an odorant were subset and pairwise compared to a null set of ORs with comparable protein sequences. F, Left, visualization of the data in 3D using homology models. Color schemes are identical those in panels A, B, and E. TMDs are indicated. Right, zoomed in view focusing on residues found directly above the conserved Y^{6.48} toggle switch residue.

FIG. 5 shows details of chemical and receptor space sampling. A, Schematic of the pS6-IP-Seq experiment. B, Top, tested odorants in chemical space colored by odor category. Bottom, molecular structures of tested odorants sorted by odor category. C, Left, the cumulative percent variance of the tested odorants in chemical space explained as a function of included PCs of molecular properties.

FIG. 6 shows details of in vitro validation. A, Volcano plots for pS6-IP-Seq results by exposure of mice to 1% (+)-carvone and 1% (−)-carvone. A red line at FDR=0.05 is drawn. ORs enriched by just (+)-odorant are colored red while ORs enriched by just (−)-odorant are colored blue. ORs enriched by both enantiomers are colored purple. B, Dose response curves of ORs displaying in vitro response to at least one of the tested carvone enantiomers. C, Volcano plots for pS6-IP-Seq results using 1M (+)-menthol and 1M (−)-menthol. D, Dose response curves of ORs displaying in vitro responses towards menthol enantiomers. E, Volcano plots for pS6-IP-Seq results using 10% (+)-2-octanol and 10% (−)-2-octanol. F, Dose response curves of ORs displaying in vitro responses towards 2-octanol enantiomers.

FIG. 7 shows details of OR response properties. A, Odorant responses determined by pS6-IP-Seq across all tested 72 odorants/concentrations visualized by heatmap. B, Left, histogram (bin size=0.05) of the distributions of OR responses across the panel of 52 odorants tested at low concentrations. Middle, zoomed in. Right, A small number of inhibitory responses (log₂FC<0 and FDR<0.05) were observed in the data. These responses were otherwise classified as nonsignificant (n=44). C, Left, distribution of the OR-molecular property pairwise Pearson correlation coefficients using the 52 odorants tested at low concentrations (bin size=0.01). Right, zoomed in.

FIG. 8 shows details of thiol property tuning. A, Tuning towards molecular properties described by the number of significant OR-molecular property associations. Molecular property nSH (number of thiol groups) is highlighted in red. B, Visualization of the average Pearson correlation tuning value for ORs that were significantly tuned toward each of the 1005 molecular properties. C, Top, tuning of individual ORs, measured by Pearson correlation, towards nSH. Bottom, the top ten ORs displaying nSH tuning. D, Raw pS6-IP-Seq differential expression data, visualized by volcano plot, with chemical structures of the tested thiol odorants. ORs that were tuned towards thiol are highlighted in red.

FIG. 9 shows details of the pairwise comparisons between odorants in response and molecular property space. A, Schematic of how pairwise distance comparisons between odorants were calculated for FIGS. 3A and 3C. B, Correspondence between odorants in response and molecular property spaces.

FIG. 10 shows details of the odor pair cross-validation for regression models. A, Schematic of how odor pair cross-validation was performed. Pairs of odorants were iteratively held-out from training. B, Results of LASSO regression using various metrics to quantify distances between odorants in response space.

FIG. 11 shows performance of optimized molecular properties in predicting receptor response similarities. A, Schematic of how “optimized” molecular properties were selected. B, Correspondence between odorants in response and “optimized” molecular property spaces. C, Results of ridge regression using various metrics to quantify distances between odorants in response space.

FIG. 12 shows results of using the XGBoost model framework. A, Using default XGBoost hyperparameters with 1811 molecular property descriptors, it was asked how well does odor molecular property similarity predict receptor response similarity. B, Results of the XGBoost models when only the 65 set of “optimized” odor molecular properties were used as predictors and odor pair cross-validation is performed.

FIG. 13 shows details of logistic regression to uncover sequence-function relationships of ORs. Data for odorants solvable by regularized logistic regression (AUROC>0.5) is shown.

FIG. 14 shows details of SVM classifiers and comparing between approaches. A, Results of using a linear SVM classifier to predict OR response likelihoods across 100 repetitions of splitting ORs into 90% training and 10% testing sets. B, Regressing convergent evolution counts by percent residue conservation reveals an anti-correlation (r=−0.54, p=6.5E-25). C, Regressing logistic model dependency, evaluated by average positional weight, by convergent evolution count reveals consistency between approaches (r=0.42, p=1.5E-14).

### DEFINITIONS

To facilitate understanding of the invention, a number of terms are defined below.

As used herein, the term “amino acid property descriptors” refers to amino acid properties of odorant receptors that are useful in determining interactions of odorant receptors with odorants. In some embodiments, the amino acid property descriptors are compared at specific positions in an alignment of known odorant receptor sequences.

As used herein, the term “physiochemical properties” refers to properties of odorants that are useful in determining interactions of odorants with odorant receptors. Examples of physiochemical properties that find use in identifying odorant receptors that are likely to interact with a given odorant include, but are not limited to, Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function −11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation-lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, and R maximal index/weighted by atomic polarizabilities.

As used herein, the term “odorant receptor” refers to odorant receptors generated from olfactory sensory neurons.

As used herein, the term “odorant receptor cell surface localization” or equivalent terms refer to the molecular transport of an odorant receptor to a cell surface membrane. Examples of cell surface localization includes, but is not limited to, localization to cilia at the tip of a dendrite, and localization to an axon terminal.

As used herein, the term “odorant receptor functional expression” or equivalent terms, refer to an odorant receptor's ability to interact with an odorant receptor ligand (e.g., an odiferous molecule).

As used herein, the term “olfactory disorder,” “olfactory dysfunction,” “olfactory disease” or similar term refers to a disorder, dysfunction or disease resulting in a diminished olfactory sensation (e.g., smell aberration). Examples of olfactory disorders, dysfunctions and/or diseases include, but are not limited to, head trauma, upper respiratory infections, tumors of the anterior cranial fossa, Kallmann syndrome, Foster Kennedy syndrome, Parkinson's disease, Alzheimer's disease, Huntington chorea, and exposure to toxic chemicals or infections. Diminished olfactory sensation is classified as anosmia—absence of smell sensation; hyposmia-decreased smell sensation; dysosmia—distortion of smell sensation; cacosmia—sensation of a bad or foul smell; and parosmia—sensation of smell in the absence of appropriate stimulus.

As used herein, the terms “subject” and “patient” refer to any animal, such as a mammal like a dog, mouse, rat, pig, cat, bird, livestock, and preferably a human. Specific examples of “subjects” and “patients” include, but are not limited to, individuals with an olfactory disorder, and individuals with olfactory disorder-related characteristics or symptoms.

As used herein, the phrase “symptoms of an olfactory disorder” and “characteristics of an olfactory disorder” include, but are not limited to, a diminished olfactory sensation (e.g., smell aberration).

The phrase “under conditions such that the symptoms are reduced” refers to any degree of qualitative or quantitative reduction in detectable symptoms of olfactory disorders, including but not limited to, a detectable impact on the rate of recovery from disease, or the reduction of at least one symptom of an olfactory disorder.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Km” as used herein refers to the Michaelis-Menton constant for an enzyme and is defined as the concentration of the specific substrate at which a given enzyme yields one-half its maximum velocity in an enzyme catalyzed reaction.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the term “purified” or “to purify” refers to the removal of contaminants from a sample. For example, odorant receptor antibodies are purified by removal of contaminating non-immunoglobulin proteins; they are also purified by the removal of immunoglobulin that does not bind an odorant receptor polypeptide. The removal of non-immunoglobulin proteins and/or the removal of immunoglobulins that do not bind an odorant receptor results in an increase in the percent of odorant receptor-reactive immunoglobulins in the sample. In another example, recombinant odorant receptor polypeptides are expressed in bacterial host cells and the polypeptides are purified by the removal of host cell proteins; the percent of recombinant odorant receptor polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein, is used to indicate a protein that does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “test compound” refers to any chemical entity, pharmaceutical, drug, and the like that can be used to treat or prevent a disease, illness, sickness, or disorder of bodily function, or otherwise alter the physiological or cellular status of a sample (e.g., odorant). Test compounds comprise both known and potential therapeutic compounds. A test compound can be determined to be therapeutic by screening using the screening methods of the present invention.

A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response,” when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, CA), chloramphenicol acetyltransferase, 3-galactosidase, alkaline phosphatase, and horse radish peroxidase.

The term “system” is used to refer to an on-line odorant receptor-odorant interaction system, an example of which is described in the present specification. The term “database” is used to refer to a data structure for storing information for use by the system, and an example of such a data structure in described in the present specification.

The term “user” refers to a person using the systems or methods of the present invention.

As used herein, the terms “processor” and “central processing unit” or “CPU” are used interchangeably and refer to a device that is able to read a program from a computer memory (e.g., ROM or other computer memory) and perform a set of steps according to the program. As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP. As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users. As used herein, the term “HTML” refers to HyperText Markup Language that is a standard coding convention and set of codes for attaching presentation and linking attributes to informational content within documents. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”). As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL. As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “in electronic communication” refers to electrical devices (e.g., computers, processors, etc.) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, infrared signals, telephone lines, etc) information to another computer or device, is in electronic communication with the other computer or device. As used herein, the term “transmitting” refers to the movement of information (e.g., data) from one location to another (e.g., from one device to another) using any suitable means.

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

### DETAILED DESCRIPTION OF THE INVENTION

The olfactory system represents one of the oldest sensory modalities in the phylogenetic history of mammals. Olfaction is less developed in humans than in other mammals such as rodents. As a chemical sensor, the olfactory system detects food and influences social and sexual behavior. The specialized olfactory epithelial cells characterize the only group of neurons capable of regeneration. Activation occurs when odiferous molecules come in contact with specialized processes known as the olfactory vesicles. Within the nasal cavity, the turbinates or nasal conchae serve to direct the inspired air toward the olfactory epithelium in the upper posterior region. This area (only a few centimeters wide) contains more than 100 million olfactory receptor cells. These specialized epithelial cells give rise to the olfactory vesicles containing kinocilia, which serve as sites of stimulus transduction.

There are three specialized neural systems are present within the nasal cavities in humans: 1) the main olfactory system (cranial nerve I), 2) trigeminal somatosensory system (cranial nerve V), 3) the nervus terminalis (cranial nerve 0). CN I mediates odor sensation. It is responsible for determining flavors. CN V mediates somatosensory sensations, including burning, cooling, irritation, and tickling. CN 0 is a ganglionated neural plexus. It spans much of the nasal mucosa before coursing through the cribriform plate to enter the forebrain medial to the olfactory tract. The exact function of the nervus terminalis is unknown in humans. The olfactory neuroepithelium is a pseudostratified columnar epithelium. The specialized olfactory epithelial cells are the only group of neurons capable of regeneration. The olfactory epithelium is situated in the superior aspect of each nostril, including cribriform plate, superior turbinate, superior septum, and sections of the middle turbinate. It harbors sensory receptors of the main olfactory system and some CN V free nerve endings. The olfactory epithelium loses its general homogeneity postnatally, and as early as the first few weeks of life metaplastic islands of respiratory-like epithelium appear. The metaplasia increases in extent throughout life. It is presumed that this process is the result of insults from the environment, such as viruses, bacteria, and toxins.

There are 6 distinct cells types in the olfactory neuroepithelium: 1) bipolar sensory receptor neurons, 2) microvillar cells, 3) supporting cells, 4) globose basal cells, 5) horizontal basal cells, 6) cells lining the Bowman's glands. There are approximately 6,000,000 bipolar neurons in the adult olfactory neuroepithelium. They are thin dendritic cells with rods containing cilia at one end and long central processes at the other end forming olfactory fila. The olfactory receptors are located on the ciliated dendritic ends. The unmyelinated axons coalesce into 40 bundles, termed olfactory fila, which are ensheathed by Schwann-like cells. The fila transverses the cribriform plate to enter the anterior cranial fossa and constitute CN I. Microvillar cells are near the surface of the neuroepithelium, but the exact functions of these cells are unknown. Supporting cells are also at the surface of the epithelium. They join tightly with neurons and microvillar cells. They also project microvilli into the mucus. Their functions include insulating receptor cells from one another, regulating the composition of the mucus, deactivating odorants, and protecting the epithelium from foreign agents. The basal cells are located near the basement membrane, and are the progenitor cells from which the other cell types arise. The Bowman's glands are a major source of mucus within the region of the olfactory epithelium.

The odorant receptors are located on the cilia of the receptor cells. Each receptor cell expresses a single odorant receptor gene. There are approximately 1,000 classes of receptors at present. The olfactory receptors are linked to the stimulatory guanine nucleotide binding protein Golf. When stimulated, it can activate adenylate cyclase to produce the second messenger cAMP, and subsequent events lead to depolarization of the cell membrane and signal propagation. Although each receptor cell only expresses one type of receptor, each cell is electrophysiologically responsive to a wide but circumscribed range of stimuli. This implies that a single receptor accepts a range of molecular entities.

The olfactory bulb is located on top of the cribriform plate at the base of the frontal lobe in the anterior cranial fossa. It receives thousands of primary axons from olfactory receptor neurons. Within the olfactory bulb, these axons synapse with a much smaller number of second order neurons which form the olfactory tract and project to olfactory cortex. The olfactory cortex includes the frontal and temporal lobes, thalamus, and hypothalamus. Although mammalian ORs were identified over 10 years ago, little is known about the selectivity of the different ORs for chemical stimuli, mainly because it has been difficult to express ORs on the cell surface of heterologous cells and assay their ligand-binding specificity (see, e.g., Mombaerts, P. (2004) Nat Rev Neurosci 5, 263-278; herein incorporated by reference in its entirety). The reason is that OR proteins are retained in the ER and subsequently degraded in the proteosome (see, e.g., Lu, M., et al., (2003) Traffic 4, 416-433; McClintock, T. S., (1997) Brain Res Mol Brain Res 48, 270-278; each herein incorporated by reference in their entireties). Despite these difficulties, extensive efforts have matched about 20 ORs with cognate ligands with various degrees of certainty (see, e.g., Bozza, T., et al., (2002) J Neurosci 22, 3033-3043; Gaillard, I., et al., (2002) Eur J Neurosci 15, 409-418; Hatt, H., et al., (1999) Cell Mol Biol 45, 285-291; Kajiya, K., et al., (2001) J Neurosci 21, 6018-6025; Krautwurst, D., et al., (1998) Cell 95, 917-926; Malnic, B., et al., (1999) Cell 96, 713-723; Raming, K., et al., (1993) Nature 361, 353-356; Spehr, M., et al., (2003) Science 299, 2054-2058; Touhara, K., et al., (1999) Proc Natl Acad Sci USA 96, 4040-4045; Zhao, H., et al., (1998) Science 279, 237-242; each herein incorporated by reference in their entirety). Adding the 20 N-terminal amino acids of rhodopsin (e.g., Rho-tag) or a foreign signal peptide to the N-terminus facilitates surface expression of some ORs in heterologous cells (see, e.g., Hatt, H., et al., (1999) Cell Mol Biol 45, 285-291; Krautwurst, D., et al., (1998) Cell 95, 917-926; each herein incorporated in their entirety). However, for most ORs, modifications do not reliably promote cell-surface expression. For example, ODR-4, which is required for proper localization of chemosensory receptors in C. elegans, has a small effect on facilitating cell-surface expression of one rat OR, but not another OR (see, e.g., Gimelbrant, A. A., et al., (2001) J Biol Chem 276, 7285-7290; herein incorporated by reference). These findings indicate that olfactory neurons have a selective molecular machinery that promotes proper targeting of OR proteins to the cell surface, but no components of this machinery have been identified (see, e.g., Gimelbrant, A. A., et al., (2001) J Biol Chem 276, 7285-7290; McClintock, T. S., and Sammeta, N. (2003) Neuroreport 14, 1547-1552; each herein incorporated by reference in their entirety).

For some GPCRs, accessory proteins are required for correct targeting to the cell surface membrane (see, e.g., Brady, A. E., and Limbird, L. E. (2002) Cell Signal 14, 297-309; herein incorporated by reference in its entirety). These proteins include NinaA for Drosophila Rhodopsin (see, e.g., Baker, E. K., et al., (1994) Embo J 13, 4886-4895; Shieh, B. H., et al., (1989) Nature 338, 67-70; each herein incorporated by reference in their entirety), RanBP2 for mammalian cone opsin (see, e.g., Ferreira, P. A., et al., (1996) Nature 383, 637-640; herein incorporated by reference in its entirety), RAMPs for the mammalian calcitonin receptor-like receptor (CRLR) (see, e.g., McLatchie, L. M., et al., (1998) Nature 393, 333-339; herein incorporated by reference in its entirety) and finally the M10 family of MHC class I proteins and beta 2 microglobulin for V2Rs, the putative mammalian pheromone receptors (see, e.g., Loconto, J., et al., (2003) Cell 112, 607-618; herein incorporated by reference in its entirety). With the exception of NinaA and RanBP2, none of these accessory proteins share any sequence homology to with each other; their only common feature is their association with the membrane. The present invention provides novel proteins (e.g., REEP1, RTP1, RTP2, RTP1-A, RTP1-B, RTP1-C, RTP1-D, RTP1-E, RTP1-A1, RTP1-D1, RTP-D2, RTP-D3, RTP1-A1-A (Chimera 1), RTP1-A1-D2 (Chimera 2), RTP1-A1-D1 (Chimera 3), RTP4-A1-A (Chimera 4), RTP4-A1-D2 (Chimera 5), and RTP4-A1-D1 (Chimera 6)) promoting OR cell surface localization and OR functional expression, and numerous compositions and methods related to these findings.

In some embodiments, the present invention provides systems and methods for identifying odorant receptor-odorant interactions. In some embodiments, the model provides an in silico method to identify candidate odorants that are likely to interact with a given odorant receptor (e.g., a known or novel odorant receptor). In some embodiments, the present invention provides methods of identifying odorants with binding properties similar to a known odorant. In other embodiments, the model provides an in silico method to identify odorant receptors that are likely to interact with a given odorant.

Accordingly, in some embodiments, provided herein is a method of identifying a ligand for an odorant receptor, comprising: a) determining the sequence of transmembrane domains 4 and 5 of the receptor; and b) identifying the receptor as likely to bind to a sulfurous odorant when one or more of a E or R amino acid at position 137, a P amino acid at position 138, A N amino acid at position 141, a C amino acid at position 148, a T amino acid at position 149, a G amino acid at position 151, a G amino acid at position 152, a A amino acid at position 153, a D amino acid at position 155, a G amino acid at position 156, a F amino acid at position 157, a M amino acid at position 158, a Y amino acid at position 195, a N amino acid at position 196, a T amino acid at position 197, a F amino acid at position 198, an M amino acid at position 199, a T, V or N amino acid at position 200, a A amino acid at position 201, a C or D amino acid at position 202, a C amino acid at position 203, a A or Y amino acid at position 205, an M amino acid at position 206, a N amino acid at position 210, a V amino acid at position 213, a L amino acid at position 214, a S amino acid at position 215, a S amino acid at position 219, a S amino acid at position 220, and an L amino acid at position 223 is present in the receptor.

Further embodiments provide a method of identifying a ligand for an odorant receptor, comprising: a) determining the sequence of transmembrane domains 4 and 5 of the receptor; and b) identifying the receptor as likely to bind to a carboxylic acid odorant when one or more of an R amino acid at position 150, a I amino acid at position 155, and a D amino acid at position 210 is present in the receptor.

Also provided is a method of identifying a ligand for an odorant receptor, comprising: a) determining the sequence of transmembrane domains 4 and 5 of the receptor; and b) identifying the receptor as likely to bind to an aldehyde acid odorant when one or more of a I or V amino acid at position 138, an R amino acid at position 139, a Y amino acid at position 141, a V amino acid at position 148, a C amino acid at position 149, a S amino acid at position 150, a T amino acid at position 151, an N amino acid at position 155, a W amino acid at position 156, a K or A amino acid at position 158, an A or P amino acid at position 195, a A amino acid at position 196, a E amino acid at position 197, an E or N amino acid at position 198, a P amino acid at position 199, a S amino acid at position 202, a V or R amino acid at position 203, a S amino acid at position 205, a C, N or V amino acid at position 209, a F amino acid at position 212, a A or G amino acid at position 215, a Y amino acid at position 220, and a H amino acid at position 224 is present in the receptor.

Additional embodiments provide a method of identifying a ligand for an odorant receptor, comprising: a) determining the sequence of transmembrane domains 4 and 5 of the receptor; and b) identifying the receptor as likely to bind to an alcohol odorant when one or more of a F, G or E amino acid at position 137, a L, P or H amino acid at position 138, a T, A or K amino acid at position 139, a R, Q or S amino acid at position 142, a L amino acid at position 143, a T or P amino acid at position 147, a S amino acid at position 148, a M or G amino acid at position 149, a A or S amino acid at position 150, a A or S amino acid at position 151, a C amino acid at position 152, a E or C amino acid at position 154, a E amino acid at position 155, a G amino acid at position 157, a H, Q, E, C, or F amino acid at position 158, a E or V amino acid at position 155, a P, K or Q amino acid at position 195, a W or M amino acid at position 197, a S or Q amino acid at position 198, a P or N amino acid at position 199, a Y amino acid at position 200, a C or Y amino acid at position 201, a G, S or F amino acid at position 202, an G, H, A or I amino acid at position 203, a V or N amino acid at position 204, a N amino acid at position 206, an A, I or F amino acid at position 207, a P amino acid at position 208, an A amino acid at position 209, a A or V amino acid at position 210, a M amino acid at position 211, an T, V, S or I amino acid at position 212, a G amino acid at position 215, a V or N amino acid at position 216, a W amino acid at position 219, a R amino acid at position 220, a F amino acid at position 222, a V amino acid at position 223, and a C amino acid at position 210 is present in the receptor.

Further provided is a method of identifying a ligand for an odorant receptor, comprising: a) determining the sequence of transmembrane domains 4 and 5 of the receptor; and b) identifying the receptor as likely to bind to a ketone odorant when one or more of a L or H amino acid at position 138, a H or R amino acid at position 141, a S or N amino acid at position 142, a M or G amino acid at position 146, a V amino acid at position 149, a M or Y amino acid at position 150, an E, W or G amino acid at position 154, a A or P amino acid at position 155, a P or W amino acid at position 156, a A amino acid at position 158, an M or P amino acid at position 195, a A amino acid at position 196, a G amino acid at position 198, a P or C amino acid at position 199, a F amino acid at position 202, an M or V amino acid at position 203, a P, M or G amino acid at position 205, a H or P amino acid at position 206, a I amino acid at position 207, a V amino acid at position 209, a T amino acid at position 211, a T amino acid at position 213, a F amino acid at position 215, a T amino acid at position 217, a Q amino acid at position 220, a M amino acid at position 223, and a N amino acid at position 224 is present in the receptor.

Additionally provided is a method of identifying a ligand for an odorant receptor, comprising: a) determining the sequence of transmembrane domains 4 and 5 of the receptor; and b) identifying the receptor as likely to bind to a pyridine or pyrazine odorant when one or more of a M amino acid at position 137, an C or A amino acid at position 138, a R amino acid at position 140, a V amino acid at position 143, a V amino acid at position 147, a G amino acid at position 148, a N amino acid at position 152, a T amino acid at position 153, a D amino acid at position 154, a Y or S amino acid at position 155, a G amino acid at position 158, an P, H or Y amino acid at position 195, an F amino acid at position 196, a S amino acid at position 197, a P amino acid at position 209, a T amino acid at position 201, a M amino acid at position 202, a F or L amino acid at position 203, a F amino acid at position 204, a Y or G amino acid at position 205 is present in said receptor, a P amino acid at position 206, an E amino acid at position 207, a W amino acid at position 208, a C amino acid at position 213, a A amino acid at position 216, and a C or L amino acid at position 219 is present in the receptor.

Certain aspects provide a method of identifying a ligand for an odorant receptor, comprising: a) determining the sequence of transmembrane domains 4 and 5 of the receptor; and b) identifying the receptor as likely to bind to ester odorant when one or more of a K amino acid at position 137, a I amino acid at position 138, a S amino acid at position 140, a G amino acid at position 148, a N amino acid at position 152, a T amino acid at position 154, a N or P amino acid at position 155, a Y amino acid at position 195, a Y amino acid at position 197, a T amino acid at position 198, a G amino acid at position 201, a S amino acid at position 202, a V amino acid at position 210, a M amino acid at position 213, a T amino acid at position 215, a G amino acid at position 219, a I amino acid at position 220, and a N amino acid at position 223 is present in the receptor.

Other embodiments provide a method of identifying a ligand for an odorant receptor, comprising: a) determining the sequence of transmembrane domains 4 and 5 of the receptor; and b) identifying the receptor as likely to bind to a thiazole or thiazoline odorant when one or more of a S amino acid at position 141, a F amino acid at position 142, a M amino acid at position 144, a C amino acid at position 151, an T amino acid at position 153, a H or D amino acid at position 154, a E amino acid at position 198, a S amino acid at position 199, a L amino acid at position 203, a K amino acid at position 205, a D or P amino acid at position 206, a I amino acid at position 211, a V amino acid at position 214, a Y amino acid at position 216, a M amino acid at position 220, a S amino acid at position 223, and a D amino acid at position 210 is present in the receptor.

In some embodiments, the one or more amino acids is two or more (e.g., 2, 3, 4, 5, 6, 7, 8, 9, 10, or all) of the amino acids.

In some embodiments, the present invention provides computer implemented systems and methods for identifying odorant receptor-odorant interactions. In some embodiments, computer implemented systems and methods generate a report of the results of the modeling methods that provides candidate odorants or odorant receptors. In some embodiments, reports rank a plurality candidate odorants or odorant receptors using a numerical or other scale (e.g., graphical or textual). In some embodiments, the report is provided over the Internet or on a computer monitor.

In some embodiments, the systems and methods of the present invention are provided as an application service provider (ASP) (e.g., can be accessed by users within a web-based platform via a web browser across the Internet; is bundled into a network-type appliance and run within an institution or an intranet; or is provided as a software package and used as a stand-alone system on a single computer).

The systems and methods described herein find use in a variety of research, clinical, and commercial uses. For example, in some embodiments, the systems and methods are utilized in research applications to identify candidate odorants or odorant receptors. In some embodiment, the identification of odorant receptor-odorant interactions find use in clinical applications (e.g., in diagnosing or treating diseases of odorant function). In some embodiments, odorants identified using the systems and methods of embodiments of the present invention find use in commercial applications (e.g., as additives in food, cosmetic, perfume, household, or industrial products).

### EXPERIMENTAL

The following examples are provided in order to demonstrate and further illustrate certain preferred embodiments and aspects of the present invention and are not to be construed as limiting the scope thereof.

### Example 1

### Methods

Phosphorylated S6 Ribosomal Capture (pS6-IP)

Mice used for pS6-IP were ˜3 weeks old, mixed sex, and littermates. Mice were killed by CO₂ asphyxiation and cervical dislocation. Olfactory tissue was rapidly dissected in Buffer B (2.5 mM HEPES KOH pH 7.4, 0.63% glucose, 100 g/mL cycloheximide, 5 mM sodium fluoride, 1 mM sodium orthovanadate, 1 mM sodium pyrophosphate, 1 mM β-glycerophosphate, in Hank's balanced salt solution). Tissue pieces were then minced in 1.35 mL Buffer C (150 mM KCl, 5 mM MgCl₂, 10 mM HEPES KOH pH 7.4, 0.100 M Calyculin A, 2 mM DTT, 100 U/mL RNAsin, 100 g/mL cycloheximide, protease inhibitor cocktail, 5 mM sodium fluoride, 1 mM sodium orthovanadate, 1 mM sodium pyrophosphate, 1 mM β-glycerophosphate) and subsequently transferred to homogenization tubes for steady homogenization at 250 rpm three times and at 750 rpm nine times at 4° C. Samples were then transferred to a 1.5 mL LoBind tube (Eppendorf 022431021) and clarified at 2000×g for 10 min at 4° C. The low-speed supernatant was transferred to a new tube on ice, and 90 L of NP40 (Sigma 11332473001) and 90 L of 1,2-diheptanoyl-sn-glycero-3-phosphocholine (DHPC, Avanti Polar Lipids 850306P, 100 mg/0.69 mL) were added to this solution. This solution was mixed and then clarified at a max speed (17,000×g) for 10 min at 4° C. The resulting high-speed supernatant was transferred to a new tube where 20 μL was saved and transferred to a tube containing 350 L buffer RLT. To the remainder of the sample, 1.3 μL of 100 μg/mL cycloheximide, 27 μL of phosphatase inhibitor cocktail (250 mM sodium fluoride, 50 mM sodium orthovanadate, 50 mM sodium pyrophosphate, 50 mM β-glycerophosphate) and 6 μL of anti-pS6 antibody (Cell Signaling D68F8) were added. The sample was gently rotated for 90 min at 4° C. To prepare beads, 100 L of beads (Invitrogen 10002D) was washed three times with 900 L of buffer A (150 mM KCl, 5 mM MgCl₂, 10 mM HEPES KOH pH 7.4, 10% NP40, 10% BSA), and once with 500 L of buffer C. Sample homogenate was added to the beads and incubated with gentle rotation for 60 min at 4° C. Following incubation, beads were washed with four times with 700 μL of buffer D (350 mM KCl, 5 mM MgCl₂, 10 mM HEPES KOH pH 7.4, 10% NP40, 2 mM DTT, 100 U/mL RNAsin, 100 μg/mL cycloheximide, 5 mM sodium fluoride, 1 mM sodium orthovanadate, 1 mM sodium pyrophosphate, 1 mM β-glycerophosphate). During the final wash, beads were moved to room temperature, wash buffer was removed, and 350 mL of buffer RLT was added. Beads were incubated in buffer RLT for 5 min at room temperature. Buffer RLT containing immunoprecipitated RNA was then eluted and stored at −80° C. until clean up using a kit (Qiagen 74004). cDNA was generated using 11 rounds of amplification with 10 ng RNA input. DNA libraries were prepared using a half-sized Nexterra XT DNA Library Preparation Kit (Illumina 15032354) protocol as per the manufacturer's guidelines. Libraries were sequenced on either HiSeq 2000/2500 (50 base pair single read mode) or NextSeq 500 (75 base pair single read mode) with 6-12 pooled indexed libraries per lane.

### RNA-Seq Alignment, Quantification, and Differential Expression Analysis

Reads were aligned against a modified GRCm38.p6 (M25) reference, in which ENSMUSG00000116179 (Olfr290) was deleted, using STAR (Dobin, A. et al. STAR: ultrafast universal RNA-seq aligner. Bioinformatics 29, 15-21, 621 doi:10.1093/bioinformatics/bts (2013)) with --outFilterMultimapNmax 10. Reads mapping to Olfr290 were inferred from ENSMUSG00000070459, with the rationale that this gene model included ENSMUSG00000116179 plus untranslated regions. Gene-level read quantification was done using RSEM (Li, B. & Dewey, C. N. RSEM: accurate transcript quantification from RNA-Seq data with or without a reference genome. BMC Bioinformatics 12, 323, doi:10.1186/1471-2105-12-323 (2011)). Differential expression analysis was performed against all genes using EdgeR (Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010)). Gene nomenclature was retrieved from BioMart (Smedley, D. et al. BioMart—biological queries made easy. BMC Genomics 10, 22, doi:10.1186/1471-2164-10-22 (2009)). Intact Olfr genes with identifiable sequences were filtered, and p-values were then re-corrected by FDR. Only ORs exhibiting odor response to at least one of the tested odorants (log₂FC>0 and FDR<0.05) were considered. A total of 555 ORs responded across the 72 different odorants at various concentrations. A total of 375 ORs were responsive to unique odorants at the lowest tested concentrations. Raw and processed RNA-Seq datasets generated as part of this study are available from NCBI GEO at accession GSE185415.

### Source of Odorants

The following odors/concentrations were used for comparing molecular properties to receptor responses: 1% 2-methyl-2-pentenal (Sigma 294667), 1% trans-cinnamaldehyde (Sigma C80687), 1% 2-heptanone (Sigma 537683; Vihani, A. et al. Semiochemical responsive olfactory sensory neurons are sexually dimorphic and plastic. Elife 9, doi:10.7554/eLife.54501 (2020)), 1% linalool (Sigma L2602), 1% ethyl butyrate (Sigma W242713), 1% guaiacol (Sigma G10903), 1% diacetyl (Sigma W237027), 1% 2-ethyl-3-methylpyrazine (Sigma W315508), 1% 2,5-dimethylpyrazine (Sigma 175420; Vihani et al., supra), 1% benzaldehyde (Sigma W212717), 1% (+)-limonene (Sigma 183164), 1% P-damascone (Sigma W324300), 1% a-pinene (Sigma W290267), 1% 2-methyl-2-thiazoline (Sigma M83406), 1% citronellol (Sigma W230915), 1% dimethyl trisulfide (Sigma W327506), 1% p-Cresol (Sigma C85751), 0.01% citral (Sigma W230316), 1 M (+)-menthol (Sigma 224464), 1 M (−)-menthol (Sigma M2780), 0.01% anisaldehyde (Sigma A88107), 1% 4-methylacetophenone (Sigma W267708), 1% methyl salicylate (Sigma W274502), 1% (+)-carvone (Sigma 22070), 1% (−)-carvone (Sigma 22060), 1% P-ionone (Sigma W259525), 1% isopropyl tiglate (Sigma W322903), 1% hexyl tiglate (Sigma W500909), 1% pyridine (Sigma 270970), 1% butyric acid (Sigma W222119), 0.01% cyclopentanethiol (Sigma W326208), 0.01% 2-butene-1-thiol (1717 CheMall Corp OR116574), 100 mM cyclopentadecanone (Sigma C111201), 1% 2-methyl-2-propanethiol (Sigma 109207), 0.01% acetophenone (Sigma W200910), 0.1% isovaleric acid (Sigma 129542), 1% isoamyl acetate (Sigma 306967), 1% ethyl tiglate (Sigma W246000), 1% heptanoic acid (Sigma W334812), 10% (+)-2-octanol (Sigma 04504), 10% (−)-2-octanol (Sigma 147990), 1% 2-hexanone (Sigma 103004), 1% 2-phenylethanol (Sigma 77861), 1% 3-methyl-1-butanethiol (Sigma W385808), 1% octanal (Sigma 05608), 1% heptanal (Sigma W254002), 1% 2,4,5-trimethylthiazole (nTMT, Sigma 219185), 100% (E)-β-Farnesene (Bedoukian P3500-90; Hu, X. S. et al. Concentration-Dependent Recruitment of Mammalian Odorant Receptors. eNeuro 7, doi:10.1523/eneuro.0103-19.2019 (2020)), 100 μM (methylthio)methanethiol (MTMT, synthesized¹⁵) 0.01% 2-sec-butyl-4,5-dihydrothiazole (SBT, synthesized; Vihani et al, supra), 77% 3,4-dehydro-exo-brevicomin (DHB, synthesized; Vihani et al, supra), and 0.01% 2,4,5-trimethyl-4,5-dihydrothiazole (TMT, synthesized; Hu et al., supra).

For logistic regression and identifying residues with predictive power towards ligand selectivity, odorants tested at the lowest concentration with at least 8 activated ORs (log₂FC>0 and FDR<0.05) were used to promote class stability. Thus, following odors were removed from consideration using logistic regression compared to above: 100 mM cyclopentadecanone, 1% 2-heptanone, 1% 2-hexanone, 1% 3-methyl-1-butanethiol, 1% α-pinene, 1% benzaldehyde, 1% β-ionone, 1% ethyl tiglate, 1% heptanoic acid, 1% hexyl tiglate, 1% isopropyl tiglate, 1% linalool, 1% methyl salicylate, 1% (+)-limonene, 1% trans-cinnamaldehyde, and 0.1% isovaleric acid. The following odors were considered at a modified concentration from above: 0.1% TMT, 0.1% acetophenone, and 10 mM MTMT.

Odorants were excluded from all analysis if no ORs were identified as responsive at the tested concentrations: 1% P-Caryophyllene (Sigma W225207¹⁵), 1% dimethyl sulfide (Sigma 274380), 1% geraniol (Sigma W250716), 1% indole (Sigma W259378), 1% (−)-dihydrocarveol (Sigma 37278), 1% (+)-dihydrocarveol (Sigma 37277), 1% propionic acid (Sigma 109797), 3 mM androstenone (Sigma 284998), or if the number of ORs identified as responsive were more than five standard deviations away from the mean: 1% 2′-hydroxyacetophenone (Sigma H18607).

### Chemical Space Estimation

To estimate chemical space, 4680 small molecules commonly found in foods and fragrances were identified from http://www.thegoodscentscompany.com/; Ravia, A. et al. A measure of smell enables the creation of olfactory metamers. Nature 588, 118-123, doi:10.1038/s41586-020-2891-7 (2020)). Three dimensional structures for these molecules and the 52 in the test odor set were then downloaded from PubChem, and 5666 molecular properties were calculated using AlvaDesc (v2.0.10). From the 5666 calculated molecular properties, 3855 were discarded because they were either not calculated for all molecules or exhibited zero variance across all molecules, leaving behind 1811 molecular descriptors. Chemical space was estimated by PCA dimensionality reduction on all molecules in R.

### Receptor Alignment and Space Estimation

Mouse ORs were aligned to one another using the MAFFT E-INS-I method with manual refinements (Katoh, K., Kuma, K., Toh, H. & Miyata, T. MAFFT version 5: improvement in accuracy of multiple sequence alignment. Nucleic Acids Res 33, 511-518, doi:10.1093/nar/gki198 (2005)). The resulting alignment file was subjected to ModelTest-NG to identify ideal amino acid substitution models (Darriba, D. et al. ModelTest-NG: A New and Scalable Tool for the Selection of DNA and Protein Evolutionary Models. Mol Biol Evol 37, 291-294, doi:10.1093/molbev/msz189 (2020)). Phylogenetic trees were generated with RAxML-NG using the JTT+I+G4 amino acid substitution model with 100 bootstraps (Kozlov, A. M., Darriba, D., Flouri, T., Morel, B. & Stamatakis, A. RAxML-NG: a fast, scalable and user-friendly tool for maximum likelihood phylogenetic inference. Bioinformatics 35, 4453-4455, doi:10.1093/bioinformatics/btz305 (2019)). Receptor pairwise similarity matrices for multidimensional scaling were generated from an alignment in which positions with amino acids in at least 60% of the receptors were considered. Receptor pairwise similarity was calculated by summing amino acid differences at each position by Grantham's amino acid distances (Grantham, R. Amino acid difference formula to help explain protein evolution. Science 185, 862-864, doi:10.1126/science.185.4154.862 (1974)). Multidimensional scaling was done in R.

### Generating Response Spectra

To generate odor response spectra, the log₂FC values of each odor-responsive OR was utilized. Each OR, r, was then centered and scaled (z-scored) by mean subtraction and standard deviation division across the odorants, o, in the test panel. The resulting matrix is denoted as \tilde{Δ}ᵣₒ. To generate property strength vectors, each molecular property, p, was z-scored across the odorants in the test panel. The resulting matrix is denoted as \tilde{P}ₒₚ. To calculate property responses and thereby property response spectra (Pearson correlation coefficients), we used the following formula:

$${Φ}_{rp} = {\sum }_{O}^{} {\tilde{Δ}}_{ro} {\tilde{P}}_{op}$$

where Φᵣₚ refers to Pearson correlation coefficients between individual receptors, r, and molecular properties, p.
To evaluate the significance of the correlation between a property and the response pattern of an OR, we used an FDR cutoff of 0.05. P-values were obtained by first calculating the t-statistic using

$$t = \frac{r \sqrt{n - 2}}{\sqrt{1 - {r}^{2}}} ,$$

where r is the correlation coefficient and n is the number of data points. The two-tailed P-value was then calculated as twice the probability a t-distributed variable exceeds t using the python scipy.stats.t.sf function. P-values were adjusted by FDR correction in R.

### Odor Distance Calculation in Property Space and Response Space

The distances between odorants in property and response space were calculated by calculating Euclidean distances, Pearson correlation coefficients, and cosine similarities between all possible unique pairs of odorants. Molecular properties and receptor response data were normalized to their respective zero mean and unit standard deviation. Correlation distances were reported as 1−r, and cosine distances were reported as 1−cos(θ).

Regression Models with Odor Pair Cross-Validation

Linear models (LASSO and ridge regression) were implemented with the glmnet package (v4.1) in R (Friedman, J., Hastie, T. & Tibshirani, R. Regularization Paths for Generalized Linear Models via Coordinate Descent. J Stat Softw 33, 1-22 (2010).). XGBoost was implemented with the xgboost module (v1.4.2) in python. Distances (Euclidean, cosine, and correlation) between each unique pair of odorants were calculated in normalized receptor response space. Then, Euclidean distances between each unique pair of odorants were calculated for each feature in normalized feature space. Regularization was then applied as either the L1 (LASSO) or L2 (ridge regression) norm. The λ loss function, which controls the number and relative contribution of selected features, was sequentially varied from zero to three by length 1000. Pearson correlation values were reported for the varied λ hyperparameters of the models by comparing model predicted response distances to true response distances. Shuffled controls consisted of using 52 fictitious odorants whose individual feature vectors were generated by resampling with replacement across the 52 test odorants.

Default XGBoost model hyperparameters were used as follows: base_score=0.5, booster-‘gbtree’, colsample_bylevel=1, colsample_bynode=1, colsample_bytree=1, gamma=0, importance_type=‘gain’, interaction_constraints=”, learning_rate=0.300000012, max_delta_step=0, max_depth=6, min_child_weight=1, missing=nan, monotone_constraints=‘( )’, n_estimators=100, num_parallel_tree=1, random_state=42, reg_alpha=0, reg_lambda=1, scale_pos_weight=1, subsample=1, tree_method=‘exact’, validate_parameters=1, and verbosity=None.

Odor pair cross-validation was performed by iteratively holding out each unique pair of odorants from the normalized 52 odor dataset (test data). Features with zero variance from the remaining 50 odor set were dropped (train data). Distances were calculated between the test data for each remaining feature (xtest) and response pattern (ytest). Train data were normalized by z-scoring independent of test data. Distances were then calculated between pairwise combinations of the 50 train odorants for each feature (xtrain) and response pattern (ytrain). Feature distances between the held-out odor pair (xtest) were then used to predict response pattern distances (ypred). Pearson correlation and mean squared error values were reported from comparing model predicted response distances (ypred) to true distances (ytest).

### Optimized Molecular Property Selection

To select a subset of molecular properties that were well represented in the data, Support Vector Machine (SVM) classifiers and regressors were utilized with linear kernels in the python sklearn.svm module (v0.24.2). Beginning with the 1811 molecular properties, those that were non-continuous (at least one zero entry, ex. molecular weight is a continuous molecular property) were first considered. Non-zero values were set to one and zero values were kept. Classifiers were trained and cross-validated across the 52 odorant molecules using the normalized 375 deorphanized receptor responses as predictors in a leave-one-out scheme. Data normalization was first performed including the test data. After removing test data, training data were normalized independently to prevent contamination. Classifier area under receiver operating characteristic (AUROC) thresholds of 0.75 were applied. Molecular properties passing this threshold were next subjected to regression with non-zero entries restored. A Pearson correlation cutoff of 0.5 was applied to finally select the 65 “optimized” molecular properties.

### Protein Sequence Analysis of ORs by Logistic Regression and SVM Classifiers

Regularized logistic regression was used to build models linking OR-protein sequence properties to OR-odor responses with the glmnet package (v4.1) in R (Friedman et al., supra). ORs were classified as responders if they exhibited log₂FC>0 and FDR<0.05 following pS6-IP-Seq and differential expression analysis. For odorants tested at multiple concentrations, the lowest concentration that activated at least 8 ORs was used to promote class stability. Predictors were generated from converting the FASTA alignment file into categorical variables reflecting the presence/absence of specific amino acids at each position.

To evaluate model performance, fitted odorants were randomly split into 90% training and 10% testing receptor sets. Each test set contained at least one responding receptor. Predictors with zero variance in the training set were dropped. The grid-search optimized a hyperparameter (setting the ratio of the L1 and L2 norms) was set by ten-fold cross-validation with ten-fold cross-validation to set the λ (loss function) value. λ values one standard error of mean greater than optimal were selected to encourage statistically identical but sparser solutions. Model weighted predictors were then used to determine the response likelihood of the test receptors. This procedure was repeated 100 times. Non-zero weights were averaged across repetitions and odorants to report positions with residues contributing predictive power towards odor selectivity. WebLogo visualizations were prepared at http://weblogo.threeplusone.com/47. SVM classifier response probabilities were calculated using the same inputs as logistic regression using 100 repetitions of 90% training (with ten-fold cross-validation for hyperparameter tuning) and 10% testing. Each repetition's response likelihoods and true outcomes were aggregated to generate a single ROC curve for a single odor, which were then combined to generate an aggregate ROC curve.

### Protein Sequence Analysis of ORs by Comparison to Convergently Evolved ORs

As an alternative strategy, a statistical evaluation of amino acid properties of ORs sharing responsiveness to an odor against convergently evolved receptors was conducted. First, responsive ORs (log₂FC>0 and FDR<0.05 from differential expression) were subset, and pairwise Grantham distances were calculated at each position to generate Grantham distance distributions within the responsive OR alignment. Pairwise comparisons between gaps were considered to have zero distance while pairwise comparisons between gaps and amino acids were considered to have the average Grantham distance across all pairwise comparisons between all ORs at that position. Null distributions were generated similarly from convergently evolved odor-unresponsive ORs. To identify convergently evolved odor-unresponsive sets of ORs, odor-specific receptors with log₂FC<0 or FDR>0.25 were first subset. Then, for each unique pairwise comparison between the odor-responsive ORs, full protein sequence Grantham distances were calculated. For each receptor in each pairwise comparison, the closest receptor was selected from the odor-unresponsive subset with the most similar absolute full protein sequence Grantham distance to the pairwise comparison. This meant, for each odor with some number of responsive receptors, there was twice as many receptors identified as convergently evolved and odor-unresponsive. Distributions were compared using the Kolmogorov-Smirnov statistical test. FDR correction was applied across all calculated P-values with a cutoff of 0.05. The number of times responding receptors displayed statistically significant deviations in the distribution of Grantham distances from the null set, at each position, was counted and summed across all odorants.

### Residue Conservation Calculation

Using the 313 length alignment file, in which each position was occupied by an amino acid in at least 60% of the responsive ORs (387 ORs that were responsive to the lowest concentration of tested odorants yielding response to at least 8 ORs each), the most common amino acid at each position was identified, termed the reduced consensus OR sequence. The percent presence of the most commonly occurring amino acid at each position was then reported as conservation percentage for said position.

### Homology Models

To build an OR homology model, previously published methods were adapted (de March, C. A., Kim, S. K., Antonczak, S., Goddard, W. A., 3rd & Golebiowski, J. G protein coupled odorant receptors: From sequence to structure. Protein Sci 24, 1543-1548, doi:10.1002/pro.2717 (2015); 49 Bushdid, C., de March, C. A., Matsunami, H. & Golebiowski, J. Numerical Models and In Vitro Assays to Study Odorant Receptors. Methods Mol Biol 1820, 77-93, doi:10.1007/978-1-4939-8609-57 (2018)). The reduced consensus OR sequence was manually re-aligned to pre-aligned sequences of the bovine rhodopsin (PDB ID 1U19), the human chemokine receptors CXCR4 (30DU) and CXCR1 (2LNL), and human adenosine A2A receptor (2YDV) using Jalview. Experimental GPCR structures of these receptors were then used as templates to build the homology model of the reduced consensus sequence with Modeller. Visualization and analysis of the homology model was done using VMD and Chimera.

### Heterologous Luciferase Assay

Hana3A cells; which stably express G_{olf}, RTP1, RTP2, and REEP1; were grown in minimum essential medium eagle (MEM; Corning 10-010-CV) containing 10% Fetal Bovine Serum (FBS; vol/vol; Gibco 16000-044), penicillin-streptomycin (Sigma-Aldrich P4333), and amphotericin B (Gibco 15290018). Cells were cultured and incubated at 37° C., 5% C02, and saturated humidity for use with the Dual-Glo Luciferase Assay (Promega E2980) (Saito, H., Kubota, M., Roberts, R. W., Chi, Q. & Matsunami, H. RTP family members induce functional expression of mammalian odorant receptors. Cell 119, 679-691, doi:10.1016/j.cell.2004.11.021 (2004); Zhuang, H. & Matsunami, H. Evaluating cell-surface expression and measuring activation of mammalian odorant receptors in heterologous cells. Nat Protoc 3, 1402-1413, doi:10.1038/nprot.2008.120 (2008). Cells were plated at 20-25% confluence on poly-D-lysine-coated 96-well plates (Corning 3843) overnight. After overnight incubation, cells were transfected with 6 mL of MEM containing 10% FBS, 0.5 μg SV40-RL (Promega E2980), 1 μg CRE-Luc (Promega E2980), 0.5 μg mouse RTP1s, 0.25 μg M3 muscarinic receptor (Li, Y. R. & Matsunami, H. Activation state of the M3 muscarinic acetylcholine receptor modulates mammalian odorant receptor signaling. Sci Signal 4, ra1, doi:10.1126/scisignal.2001230 (2011)), 0.5 μg of Rho-tagged receptor plasmid DNA, and 20 μg Lipofectamine 2000 (Invitrogen 11668019) per plate. Transfection medium was divided equally among the wells so that each OR-odorant combination could be conducted in triplicates. The following day, cells were incubated with 25 μL of odorant solution diluted in CD-293 (Gibco 11913-019) containing 30 μM CuCl₂ (Sigma-Aldrich C-6641) and 2 mM glutamine (Gibco 25030-081) for 3.5 hours. cAMP-driven firefly Luciferase luminescence (Luc) was used to assess OR activation, and SV40-driven Renilla Luciferase luminescence (Ren) was used to control for variation in cell viability within wells. Cell luminescence was read by a POLARstar OPTIMA (BMG Labtech) luminometer, and normalized response values were calculated using the formula (Luc-400)/(Ren-400). ORs were considered responsive in vitro if ANOVA p-value was <0.05 and ANOVA with post-hoc Dunnet's test correction p-adjusted was <0.05 for at least 2 of the tested odor concentrations using the R package DescTools (v0.99.42). Log-logistic 4-parameter dose response curves were fit to the data using the R package dre (v3.0-1). In vitro responses were compared to in vivo responses by subtracting mean ligand-independent activity (luciferase values of ORs with no odor stimulation) from each of the ligand stimulated data points and summing. Scaled summed (+)-enantiomer responses were divided by scaled summed (−)-enantiomer responses and log₂ transformed for comparison to log₂FC (+)/(−) in vivo enrichments.

### Results

### Estimation of Chemical and Receptor Space Sampling

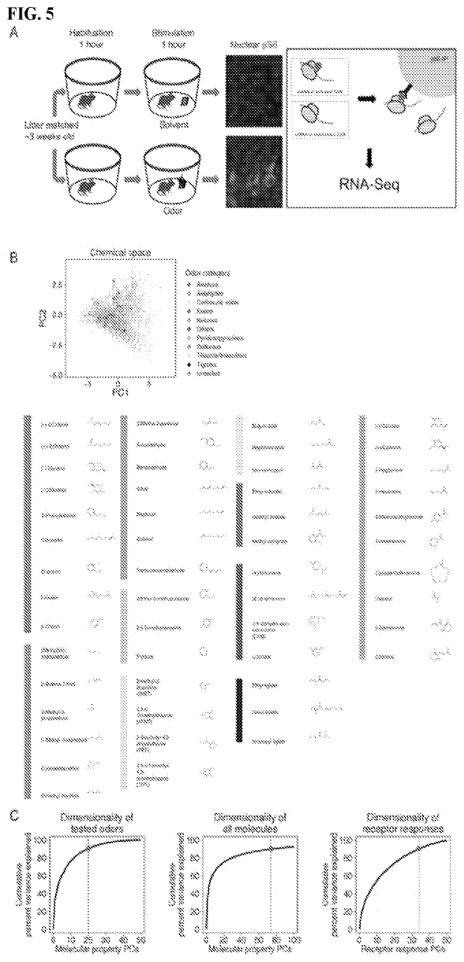

First, ORs activated by a set of 61 odorants at various concentrations were determined by leveraging pS6-IP-Seq. Immunoprecipitation of phosphorylated ribosomes from activated neurons followed by associated mRNA profiling by RNA-Seq, and differential expression analysis, enabled us to identify ORs expressed by OSNs activated by specific odorants (FIG. 5A) (Jiang, Y. et al. Molecular profiling of activated olfactory neurons identifies odorant receptors for odors in vivo. Nat Neurosci 18, 1446-1454, doi:10.1038/nn.4104 (2015); Hu et al., supra; Vahani et al., supra). ORs were considered odor-responsive if enrichment values (log₂FC) were positive with a false discovery rate (FDR)<0.05. Considering all odorants at all tested concentrations, this approach deorphanized a total of 555 ORs across 72 conditions. Considering unique odorants yielding at least one activated OR at the lowest tested concentration, this approach deorphanized a total of 375 ORs across 52 odorants.

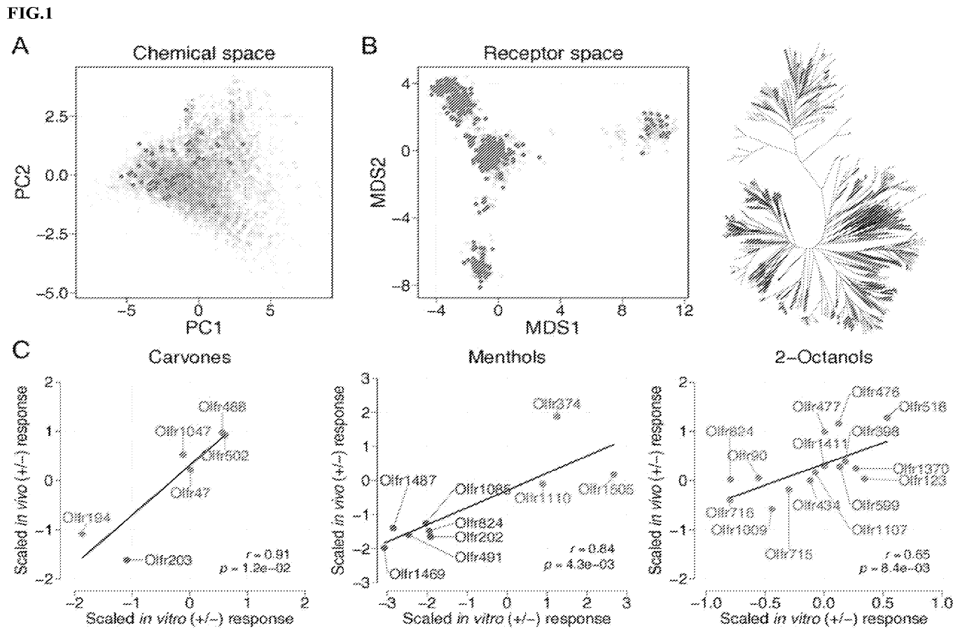

To examine the bias in the odorant set, an 1811-dimensional (1811D) space was built in which each dimension represented a molecular property descriptor (Saito, H., Chi, Q., Zhuang, H., Matsunami, H. & Mainland, J. D. Odor coding by a Mammalian receptor repertoire. Sci Signal 2, ra9, doi:10.1126/scisignal.2000016 (2009)), such as molecular weight, number of atoms, or aromatic ratio, parameterizing the physical-chemical properties of the odor molecule. The 52 uniquely tested odorants were plotted together with 4680 other small molecules (Ravia, A. et al. A measure of smell enables the creation of olfactory metamers. Nature 588, 118-562 123, doi:10.1038/s41586-020-2891-7 (2020)) and other small molecules commonly found in foods and fragrances in this 1811D space to construct a chemical space consisting of a total of 4732 small molecules. Visualization of the first two principal components (PCs) did not reveal any obvious segregation of the test odorants, suggesting a broad sampling of chemical space by the test odor panel (FIG. 1A, FIG. 5B-C). To examine bias in the resulting deorphanized OR cohort, pairwise OR Grantham distances (Grantham, R. Amino acid difference formula to help explain protein evolution. Science 185, 862-864, doi:10.1126/science.185.4154.862 (1974)), an index of amino acid similarity were computed and the results visualized using multidimensional scaling (MDS). Examination of the first two MDS coordinates did not reveal any obvious segregation of the deorphanized 375 ORs, suggesting a broad sampling of receptor space (FIG. 1B).

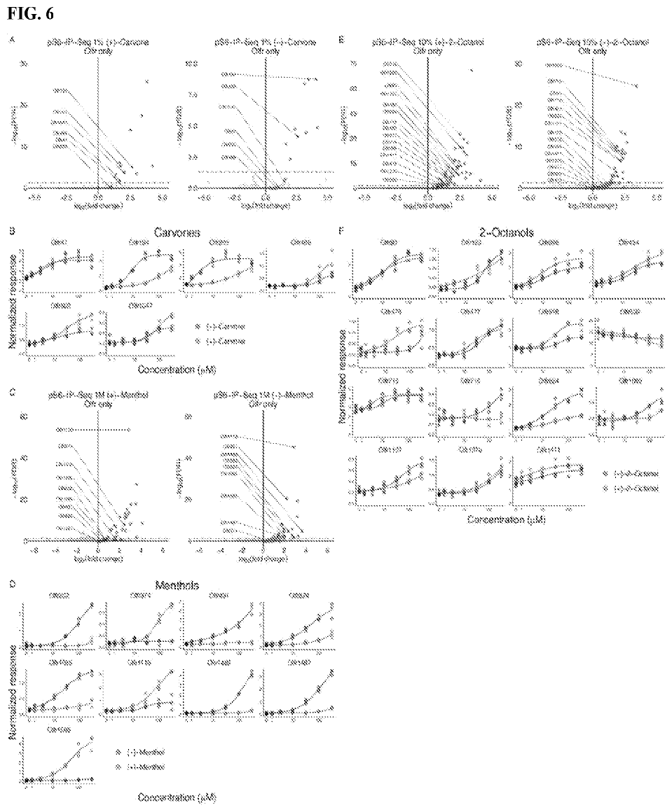

To validate the receptor specificity of the pS6-IP-Seq dataset, enantiomers (carvones, menthols, and 2-octanols) were selected for in vitro testing. ORs responsive to tested enantiomers were transiently expressed in Hana3A cells and challenged with individual odorants to generate dose response curves. Comparison of in vitro responses to in vivo responses revealed the data to be highly correlated (carvones r=0.91, p=1.2E-2; menthols r=0.84, p=4.3E-3; 2-octanols r=0.65, p=8.3E-3). Altogether, these results substantiated the pS6-IP-Seq dataset and yielded confidence that the pS6-IP-Seq strategy provides an index of receptor selectivity even amongst structurally similar odorants (FIG. 1C, FIG. 6A-F).

### Describing Receptor Tuning

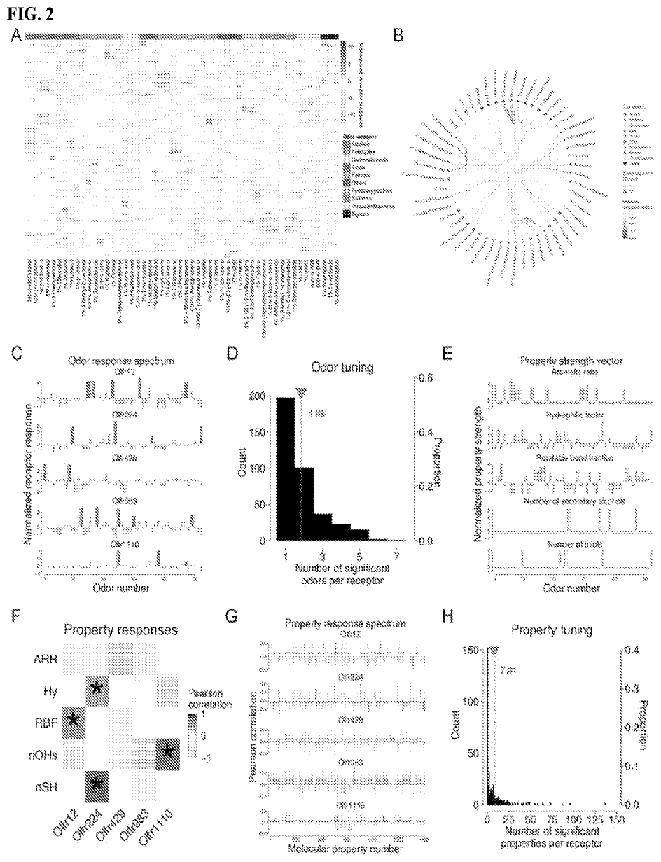

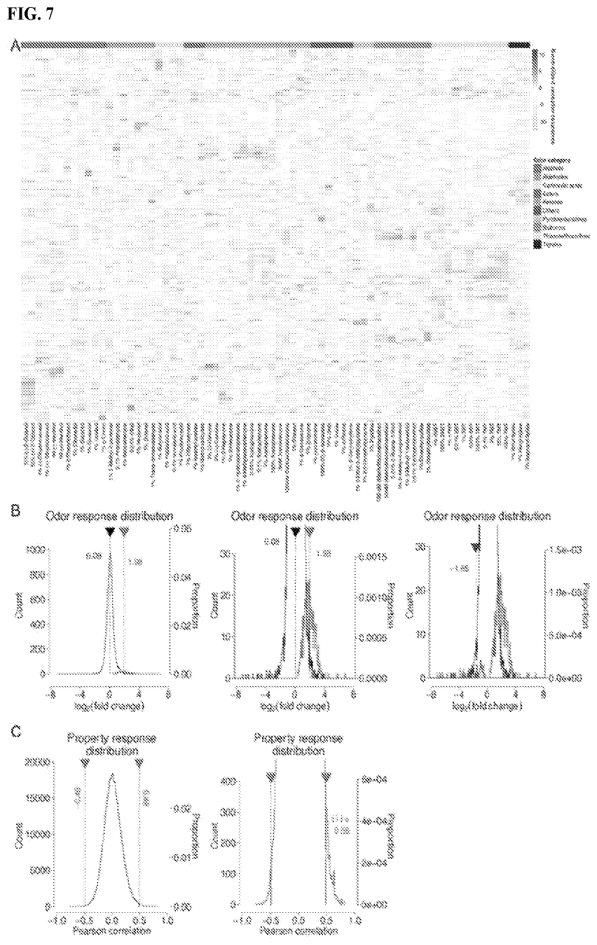

Having broadly sampled chemical and receptor spaces, the relative responses of individual receptors to the test odor panel were quantified. Individual receptors displayed unique response profiles across the odorants. Examining receptor tuning did not reveal a bimodal distribution of narrowly and broadly tuned receptors, but rather a continuum of tuning breadths with an average of 1.85 cognate odorants per significantly responding receptor (FIG. 2A-D, FIG. 7A-B).

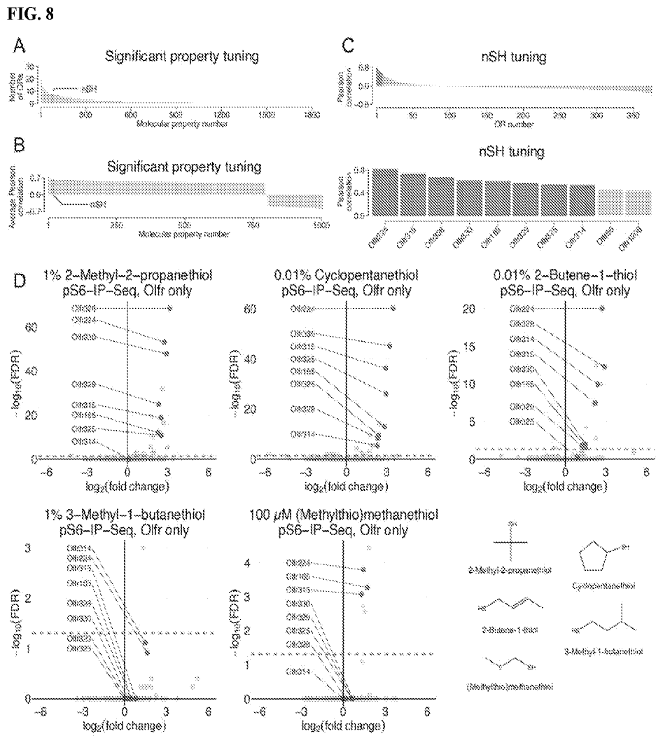

To describe the tuning of ORs towards specific molecular properties, property strength vectors (PSVs) were generated for each of the molecular descriptors (FIG. 2E) (Chae, H. et al. Mosaic representations of odors in the input and output layers of the mouse olfactory bulb. Nat Neurosci 22, 1306-1317, doi:10.1038/s41593-019-0442-z (2019)). The responsiveness of each OR to each molecular property was then characterized as a Pearson's correlation between the odor response spectrum and the values taken by the PSV across the 52 odorants tested (FIG. 2F). The array of such correlations (hereby termed property response spectrum) taken across the molecular property descriptor set defined the molecular receptive range and property tuning of each OR (FIG. 2G). For example, several ORs that displayed robust responses towards thiol odorants yielded tuning towards the “number of thiol groups” molecular property (FIG. 8A-D). The number of properties that single receptors responded to significantly (FDR<0.05) varied from receptor to receptor with a range of 0 to 136 (mean=7.91, median=2). Indeed, the majority of deorphanized ORs (223/375) displayed significant correlations to at least one of the molecular property descriptors (FIG. 2H). Within the subset of significant OR response-property pairs (2967/679125), correlations spanned both negative (−0.77 to −0.49) and positive (0.49 to 0.82) values with an absolute average of 0.55 (FIG. 7C).

### Odor Molecular Properties are Informative of Odor Response Patterns

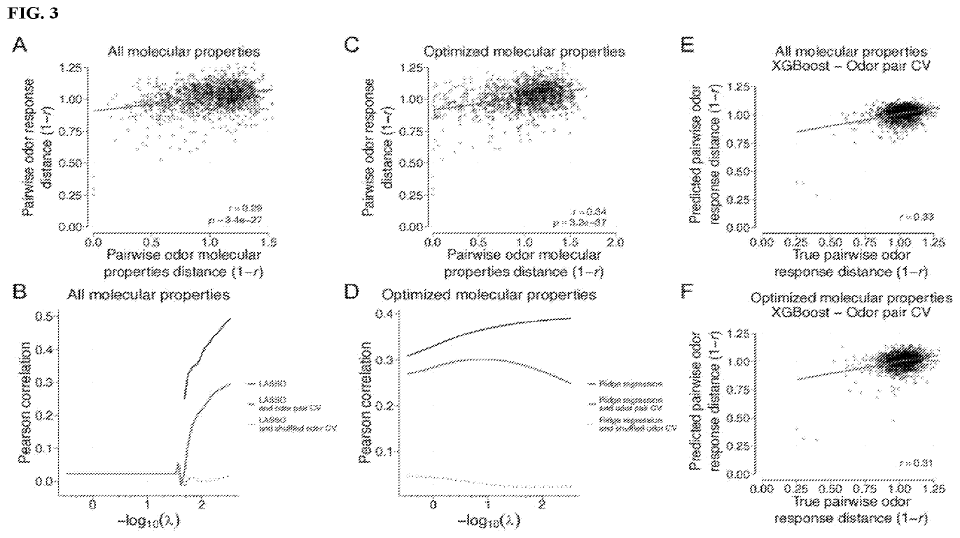

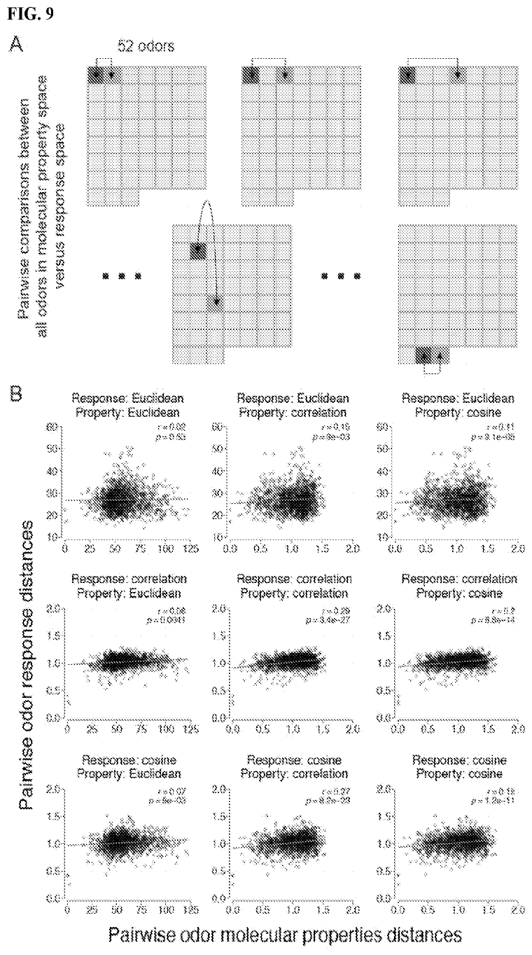

Having identified receptor responses to a large and diverse set of odorants, the effectiveness of using odor molecular properties to predict receptor responses via similarities was next determined (Saito, H., Chi, Q., Zhuang, H., Matsunami, H. & Mainland, J. D. Odor coding by a Mammalian receptor repertoire. Sci Signal 2, ra9, doi:10.1126/scisignal.2000016 (2009); Chae et al., supra; Pashkovski, S. L. et al. Structure and flexibility in cortical representations of odour space. Nature 583, 253-258, doi:10.1038/s41586-020-2451-1 (2020). To describe the similarity between odorants in molecular property space, distances of normalized property strength values between odorant pairs were calculated. To represent odor similarity in OR response space, pairwise distances between normalized receptor responses were calculated. Linear regression between odorant similarity distances and response similarity distances revealed a significant relationship (r=0.29, p=3.4E-27; FIG. 3A, FIG. 9A-B), indicating odor molecular properties were informative of receptor response patterns.

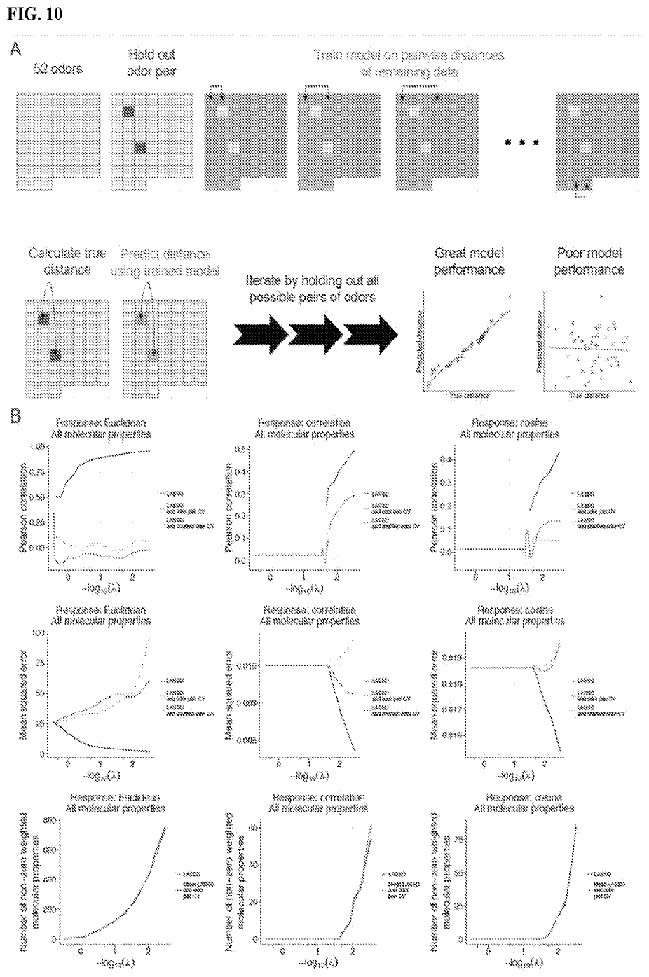

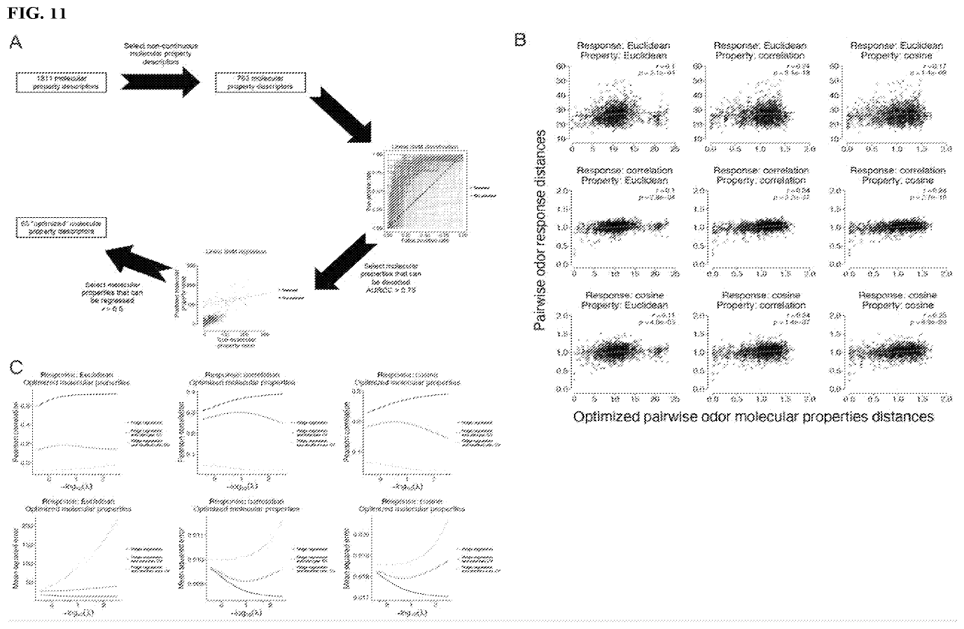

The possibility that a subset of the molecular property descriptors may be better able to relate odor molecular property similarities to receptor response similarities was next considered. To test this possibility, a sparse regression was built and used to perform feature selection using the Least Absolute Shrinkage and Selection Operator (LASSO). By varying the LASSO loss function (λ) to influence the number and relative contribution of the selected molecular properties, improved correlations with increasing numbers of weighted molecular properties were observed. By performing odor pair cross-validation, it was observed that parsimonious combinations of odor molecular properties selected by LASSO yielded positive predictive abilities (optimal correlation distance odor pair cross-validation r=0.30, FIG. 3B, FIG. 10A-B). To complement these findings, an “optimized” set of 65 molecular properties; which included descriptions of aromaticity, functional group, and molecular geometry; that could be individually linearly decoded and regressed from OR response patterns alone was selected (FIG. 11A). Using ridge regression with the “optimized” set of 65 molecular properties, it was possible to predict response similarities from molecular property similarities (optimal correlation distance odor pair cross-validation r=0.30, FIG. 3C-D, FIG. 11B-C). Altogether, we interpreted these “optimized” set of 65 molecular property descriptors as both, being capable of explaining OR response variance, and contributing to the natural statistics of odorants.

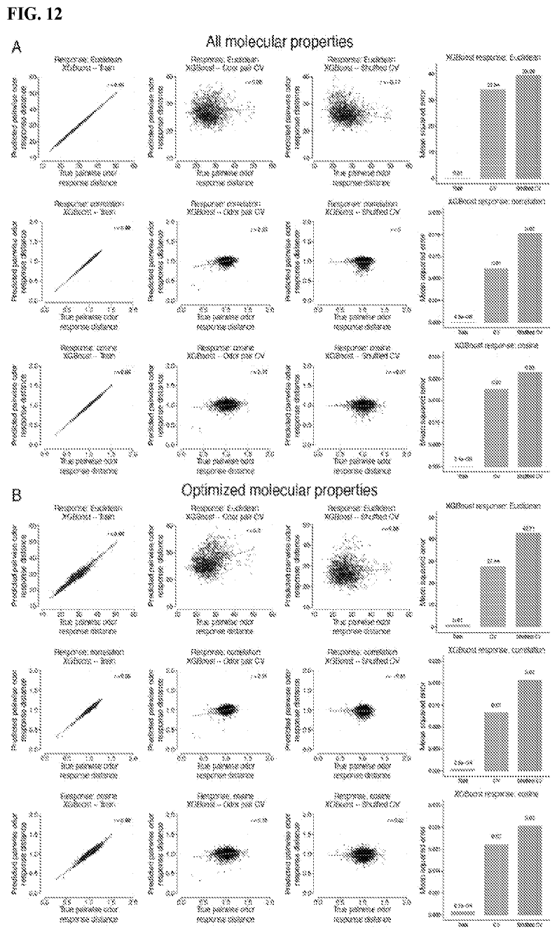

To further validate the predictive abilities of molecular properties and our molecular property optimization, a feed-forward non-linear model (XGBoost) was trained and cross-validated. In the first cross-validation scheme, odor-pair cross-validation was performed using all calculated molecular properties as predictors. In the second, molecular properties were limited to the “optimized” set. In both cross-validation schemes, predicting response similarities from molecular properties outperformed shuffled controls (FIG. 3E-F, FIG. 12A-B). Altogether, these results show that odor responses can be explained in part by combinations of molecular property descriptors.

### Specific Receptor Residues Predict Ligand Selectivity of ORs

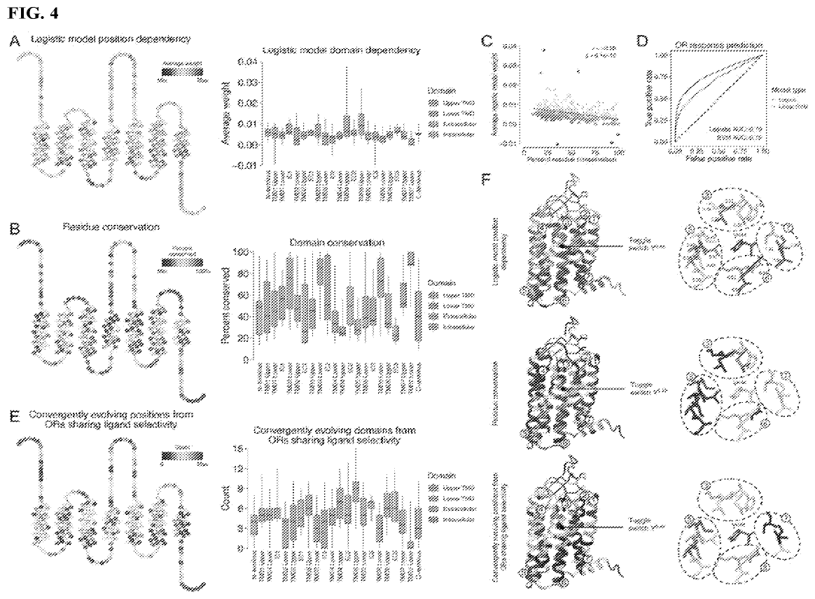

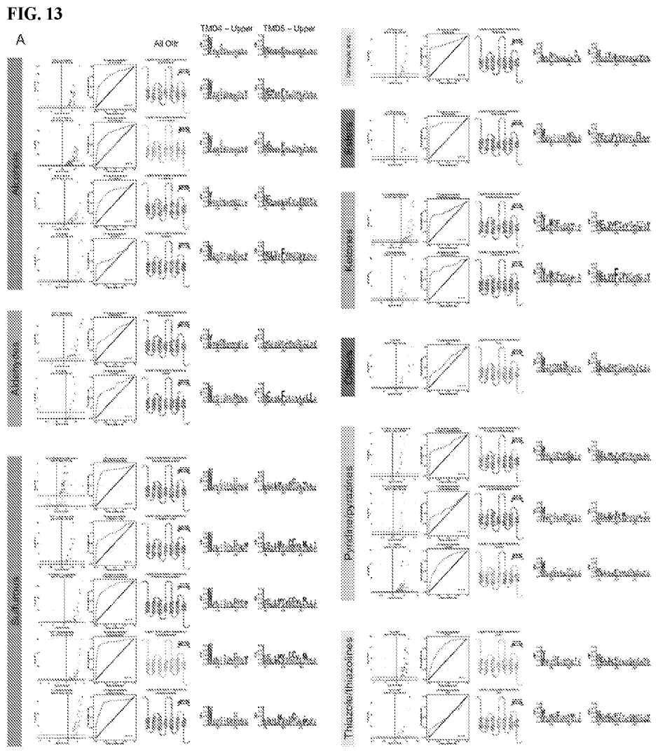

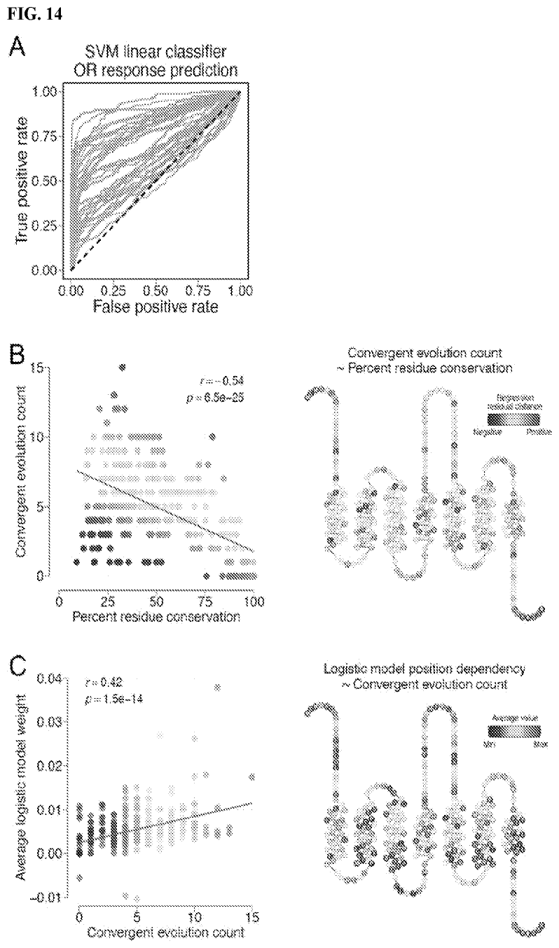

Comprehensive identification of ORs responsive to many odorants led to a search for generalizable relationships between odorants, ORs, and receptor residues (Jiang, Y. et al. Molecular profiling of activated olfactory neurons identifies odorant receptors for odors in vivo. Nat Neurosci 18, 1446-1454, doi:10.1038/nn.4104 (2015); Hu, X. S. et al. Concentration-Dependent Recruitment of Mammalian Odorant Receptors; eNeuro 7, doi:10.1523/eneuro.0103-9.2019 (2020)). To do so, logistic models were built using aligned ORs. For each odor fit with a regularized logistic model, receptors were randomly split into 90% training and 10% testing sets for 100 repetitions. Iterating this process over the set of tested odorants identified a series of weighted positions, harboring amino acids with predictive power, occurring primarily at the upper halves of the fourth and fifth transmembrane domains (TMDs) (FIG. 4A). For example, visualizing amino acids occurring at these positions identified an enrichment of cysteine and methionine residues in TMD5 amongst ORs responding to sulfurous odorants (FIG. 13A). Regressing the average weight assigned to each position, from odorants solvable by logistic regression (area under receiver operating characteristic curve, AUROC>0.5), by percent conservation revealed an anti-correlation (r=−0.38,p=6.1E-12, FIG. 4B-C) (Buck, L. & Axel, R. A novel multigene family may encode odorant receptors: a molecular basis for odor recognition. Cell 65, 175-187, doi:10.1016/0092-8674(91)90418-x (1991)). Using a Support Vector Machine (SVM) classifier, with a linear kernel, led to similar predictions regarding the response likelihoods of held-out ORs (Logistic regression AUROC=0.70, Linear SVM AUROC=0.78, FIG. 4D, FIG. 14A).

The massive expansion and rapid evolution of the OR gene family posits opportunities for the convergent evolution of distantly related ORs to evolve odorant selectivity independently. To search for receptor sequence positions exhibiting convergent evolution, it was asked if ORs sharing response to an odorant possessed positions harboring amino acids with physical-chemical properties, measured by Grantham's distance (Grantham, R. Amino acid difference formula to help explain protein evolution. Science 185, 862-864, doi:10.1126/science.185.4154.862 (1974)), which deviated from comparable but odor-unresponsive ORs. Iterating over the set of tested odorants, this analysis identified a series of poorly conserved positions (r=−0.54, p=6.5E-25, FIG. 4E, FIG. 14B), especially localized to the upper half of TMD5. Regressing the average weight assigned to each position, via regularized logistic regression, by the number of times each position displayed convergent evolution, revealed similar findings between the two approaches (r=0.42, p=1.5E-14, FIG. 14C). The localization of these positions, and those identified by logistic models, was consistent with a region implicated in ligand binding in other class A GPCRs (Zhou, Q. et al. Common activation mechanism of class A GPCRs. Elife 8, doi:10.7554/eLife.50279 (2019); Liapakis, G. et al. The forgotten serine. A critical role for Ser-2035.42 in ligand binding to and activation of the beta 2-adrenergic receptor. J Biol Chem 275, 37779-37788, doi:10.1074/jbc.M002092200 (2000); Strader, C. D., Candelore, M. R., Hill, W. S., Sigal, I. S. & Dixon, R. A. Identification of two serine residues involved in agonist activation of the beta-adrenergic receptor. J Biol Chem 264, 13572-13578 (1989); Katritch, V. & Abagyan, R. GPCR agonist binding revealed by modeling and crystallography; Trends Pharmacol Sci 32, 637-643, doi:10.1016/j.tips.2011.08.001 (2011); Munk, C., Harpsoe, K., Hauser, A. S., Isberg, V. & Gloriam, D. E. Integrating structural and mutagenesis data to elucidate GPCR ligand binding. Curr Opin Pharmacol 30, 51-58, doi:10.1016/j.coph.2016.07.003 (2016); Surgand, J. S., Rodrigo, J., Kellenberger, E. & Rognan, D. A chemogenomic analysis of the transmembrane binding cavity of human G-protein-coupled receptors. Proteins 62, 509-538, doi:10.1002/prot.20768 (2006)). Altogether these results show the odorant selectivity of ORs are in part explained by convergently evolving residues occurring at a common site of poorly conserved residues within the TMDs.

To visualize the results of the analyses in 3D, an OR homology model was built. Focusing on the conserved “toggle switch” Y⁶⁴⁸ residue previously reported to reside at the bottom of the ligand-binding cavity of other class A GPCRs (Strader et al., supra; Shi, L. et al. Beta2 adrenergic receptor activation. Modulation of the proline kink in transmembrane 6 by a rotamer toggle switch. J Biol Chem 277, 40989-40996, doi:10.1074/jbc.M206801200 (2002); Eddy, M. T. et al. Allosteric Coupling of Drug Binding and Intracellular Signaling in the A2A Adenosine Receptor. Cell 172, 68-80 e12, doi:10.1038/nbt.4096 (2018); de March, C. A. et al. Conserved Residues Control Activation of Mammalian G Protein-Coupled Odorant Receptors. J Am Chem Soc 137, 8611-8616, doi:10.1021/jacs.5b04659 (2015)), nearby residues in the upper halves of TMD3, TMD5, and TMD6 were consistently observed as exhibiting heavy weights in the logistic models, poor conservation, and convergent evolution, implying a canonical cavity for odorant binding across the tested odorants. Altogether, these results are consistent with the idea that few mutations within the ligand binding site of ORs can broadly reconfigure chemical tuning, a feature that is likely to have facilitated the rapid evolution of receptors with distinct ligand specificities.

### INCORPORATION BY REFERENCE

The entire disclosure of each of the patent documents and scientific articles referred to herein is incorporated by reference for all purposes.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of identifying a ligand for an odorant receptor, comprising:
a) determining the sequence of transmembrane domains 4 and 5 of said receptor; and
b) identifying said receptor as likely to bind to a sulfurous odorant when one or more of a E or R amino acid at position 137, a P amino acid at position 138, A N amino acid at position 141, a C amino acid at position 148, a T amino acid at position 149, a G amino acid at position 151, a G amino acid at position 152, a A amino acid at position 153, a D amino acid at position 155, a G amino acid at position 156, a F amino acid at position 157, a M amino acid at position 158, a Y amino acid at position 195, a N amino acid at position 196, a T amino acid at position 197, a F amino acid at position 198, an M amino acid at position 199, a T, V or N amino acid at position 200, a A amino acid at position 201, a C or D amino acid at position 202, a C amino acid at position 203, a A or Y amino acid at position 205, an M amino acid at position 206, a N amino acid at position 210, a V amino acid at position 213, a L amino acid at position 214, a S amino acid at position 215, a S amino acid at position 219, a S amino acid at position 220, and an L amino acid at position 223 is present in said receptor.

2. A method of identifying a ligand for an odorant receptor, comprising:
a) determining the sequence of transmembrane domains 4 and 5 of said receptor; and
b) identifying said receptor as likely to bind to a carboxylic acid odorant when one or more of an R amino acid at position 150, a I amino acid at position 155, and a D amino acid at position 210 is present in said receptor.

3. A method of identifying a ligand for an odorant receptor, comprising:
a) determining the sequence of transmembrane domains 4 and 5 of said receptor; and
b) identifying said receptor as likely to bind to an aldehyde acid odorant when one or more of a I or V amino acid at position 138, an R amino acid at position 139, a Y amino acid at position 141, a V amino acid at position 148, a C amino acid at position 149, a S amino acid at position 150, a T amino acid at position 151, an N amino acid at position 155, a W amino acid at position 156, a K or A amino acid at position 158, an A or P amino acid at position 195, a A amino acid at position 196, a E amino acid at position 197, an E or N amino acid at position 198, a P amino acid at position 199, a S amino acid at position 202, a V or R amino acid at position 203, a S amino acid at position 205, a C, N or V amino acid at position 209, a F amino acid at position 212, a A or G amino acid at position 215, a Y amino acid at position 220, and a H amino acid at position 224 is present in said receptor.

4. A method of identifying a ligand for an odorant receptor, comprising:
a) determining the sequence of transmembrane domains 4 and 5 of said receptor; and
b) identifying said receptor as likely to bind to an alcohol odorant when one or more of a F, G or E amino acid at position 137, a L, P or H amino acid at position 138, a T, A or K amino acid at position 139, a R, Q or S amino acid at position 142, a L amino acid at position 143, a T or P amino acid at position 147, a S amino acid at position 148, a M or G amino acid at position 149, a A or S amino acid at position 150, a A or S amino acid at position 151, a C amino acid at position 152, a E or C amino acid at position 154, a E amino acid at position 155, a G amino acid at position 157, a H, Q, E, C, or F amino acid at position 158, a E or V amino acid at position 155, a P, K or Q amino acid at position 195, a W or M amino acid at position 197, a S or Q amino acid at position 198, a P or N amino acid at position 199, a Y amino acid at position 200, a C or Y amino acid at position 201, a G, S or F amino acid at position 202, an G, H, A or I amino acid at position 203, a V or N amino acid at position 204, a N amino acid at position 206, an A, I or F amino acid at position 207, a P amino acid at position 208, an A amino acid at position 209, a A or V amino acid at position 210, a M amino acid at position 211, an T, V, S or I amino acid at position 212, a G amino acid at position 215, a V or N amino acid at position 216, a W amino acid at position 219, a R amino acid at position 220, a F amino acid at position 222, a V amino acid at position 223, and a C amino acid at position 210 is present in said receptor.

5. A method of identifying a ligand for an odorant receptor, comprising:
a) determining the sequence of transmembrane domains 4 and 5 of said receptor; and
b) identifying said receptor as likely to bind to a ketone odorant when one or more of a L or H amino acid at position 138, a H or R amino acid at position 141, a S or N amino acid at position 142, a M or G amino acid at position 146, a V amino acid at position 149, a M or Y amino acid at position 150, an E, W or G amino acid at position 154, a A or P amino acid at position 155, a P or W amino acid at position 156, a A amino acid at position 158, an M or P amino acid at position 195, a A amino acid at position 196, a G amino acid at position 198, a P or C amino acid at position 199, a F amino acid at position 202, an M or V amino acid at position 203, a P, M or G amino acid at position 205, a H or P amino acid at position 206, a I amino acid at position 207, a V amino acid at position 209, a T amino acid at position 211, a T amino acid at position 213, a F amino acid at position 215, a T amino acid at position 217, a Q amino acid at position 220, a M amino acid at position 223, and a N amino acid at position 224 is present in said receptor.

6. A method of identifying a ligand for an odorant receptor, comprising:
a) determining the sequence of transmembrane domains 4 and 5 of said receptor; and
b) identifying said receptor as likely to bind to a pyridine or pyrazine odorant when one or more of a M amino acid at position 137, an C or A amino acid at position 138, a R amino acid at position 140, a V amino acid at position 143, a V amino acid at position 147, a G amino acid at position 148, a N amino acid at position 152, a T amino acid at position 153, a D amino acid at position 154, a Y or S amino acid at position 155, a G amino acid at position 158, an P, H or Y amino acid at position 195, an F amino acid at position 196, a S amino acid at position 197, a P amino acid at position 209, a T amino acid at position 201, a M amino acid at position 202, a F or L amino acid at position 203, a F amino acid at position 204, a Y or G amino acid at position 205 is present in said receptor, a P amino acid at position 206, an E amino acid at position 207, a W amino acid at position 208, a C amino acid at position 213, a A amino acid at position 216, and a C or L amino acid at position 219 is present in said receptor.

7. A method of identifying a ligand for an odorant receptor, comprising:
a) determining the sequence of transmembrane domains 4 and 5 of said receptor; and
b) identifying said receptor as likely to bind to an ester odorant when one or more of a K amino acid at position 137, a I amino acid at position 138, a S amino acid at position 140, a G amino acid at position 148, a N amino acid at position 152, a T amino acid at position 154, a N or P amino acid at position 155, a Y amino acid at position 195, a Y amino acid at position 197, a T amino acid at position 198, a G amino acid at position 201, a S amino acid at position 202, a V amino acid at position 210, a M amino acid at position 213, a T amino acid at position 215, a G amino acid at position 219, a I amino acid at position 220, and a N amino acid at position 223 is present in said receptor.

8. A method of identifying a ligand for an odorant receptor, comprising:
a) determining the sequence of transmembrane domains 4 and 5 of said receptor; and
b) identifying said receptor as likely to bind to a thiazole or thiazoline odorant when one or more of a S amino acid at position 141, a F amino acid at position 142, a M amino acid at position 144, a C amino acid at position 151, an T amino acid at position 153, a H or D amino acid at position 154, a E amino acid at position 198, a S amino acid at position 199, a L amino acid at position 203, a K amino acid at position 205, a D or P amino acid at position 206, a I amino acid at position 211, a V amino acid at position 214, a Y amino acid at position 216, a M amino acid at position 220, a S amino acid at position 223, and a D amino acid at position 210 is present in said receptor.

9. The method of any of the preceding claims, wherein said one or more amino acids is two or more amino acids.

10. The method of any of the preceding claims, wherein said one or more amino acids is five or more amino acids.

11. The method of any of the preceding claims, wherein said one or more amino acids is all of said amino acids.

12. The method of any of the preceding claims, wherein said method is performed in silico.

13. The method of any of the preceding claims, further comprising the step of displaying the results of said method on a computer screen.

14. The method of any of the preceding claims, further comprising the step of screening odorants and odorant receptor pairs identified in said method using an vitro assay.
